(19) United States
(12) Patent Application Publication
(10) Pub. No.: US 2021/0151188 A1
(43) Pub. Date: May 20, 2021

(54) Eating Disorder Diagnosis

(71) Applicant: Mandometer AB, Huddinge (SE)

(72) Inventors: Cecilia Bergh, Stockholm (SE); Per Södersten, Stockholm (SE); Ulf Brodin, Stockholm (SE); Modjtaba Zandian, Stockholm (SE); Michael Leon, San Juan Capistrano, CA (US)

(21) Appl. No.: 17/085,554

(22) Filed: Oct. 30, 2020

Related U.S. Application Data
(60) Provisional application No. 62/936,798, filed on Nov. 18, 2019.

(51) Int. Cl. G16H 50/20; G16H 10/20; G16H 50/70; G16H 70/60; G16H 20/60; G16H 20/70; G16H 15/00; G16H 70/20; A61B 5/00

(52) U.S. Cl. CPC G16H 50/20; G16H 10/20; G16H 50/70; G16H 70/60; A61B 5/742; G16H 20/70; G16H 15/00; G16H 70/20; A61B 5/486; G16H 20/60

(57) ABSTRACT

Novel tools and techniques are provided for implementing eating disorder diagnosis based on analysis of patient responses to a set of questions having closed-ended answer options. In various embodiments, a computing system might autonomously determine a diagnosis of whether the first patient has an eating disorder, based on logistic regression analysis of a first set of patient responses to the set of questions. If so, the computing system might autonomously identify a first set of weighted values and a second set of weighted values each corresponding to each of the received first set of patient responses from the first patient; might autonomously calculate a first probability of diagnosis of a first eating disorder and a second probability of diagnosis of a second eating disorder; might autonomously identify which eating disorder the first patient is likely to have; and might display the identified eating disorder.

### CROSS-REFERENCES TO RELATED APPLICATIONS

This application claims priority to U.S. Patent Application Ser. No. 62/936,798 (the “'798 Application”), filed Nov. 18, 2019 by Cecilia Bergh et al. (attorney docket no. 1115.04PR), entitled, “Eating Disorder Diagnosis,” the disclosure of which is incorporated herein by reference in its entirety for all purposes.

This application may be related to U.S. patent application Ser. No. 12/412,434 (the “'434 Application”; U.S. Pat. No. 10,332,054), filed Mar. 27, 2009 by Cecilia Bergh et al. (attorney docket no. 1115.02), entitled, “Method, Generator Device, Computer Program Product and System for Generating Medical Advice,” which claims priority to SE Application No. 0900156-1, filed Feb. 9, 2009, by Cecilia Bergh, the disclosure of which is incorporated herein by reference in its entirety for all purposes. This application may also be related to U.S. Patent Application Ser. No. 62/851,238 (the “'238 Application”), filed May 22, 2019 by Cecilia Bergh et al. (attorney docket no. 1115.03PR), entitled, “Method and System for Implementing Risk Assessment for Suicide and Treatment Based on Interaction with Virtual Clinician, Food Intake Tracking, and/or Satiety Determination,” the disclosure of which is incorporated herein by reference in its entirety for all purposes.

The respective disclosures of these applications/patents (which this document refers to collectively as the “Related Applications”) are incorporated herein by reference in their entirety for all purposes.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### FIELD

The present disclosure relates, in general, to methods, systems, and apparatuses for implementing eating disorder diagnosis, and, more particularly, to methods, systems, and apparatuses for implementing eating disorder diagnosis based on analysis of patient responses to a set of questions having closed-ended answer options.

### BACKGROUND

Traditionally, to diagnose eating disorders in subjects or patients, each patient must be interviewed by a trained clinician or healthcare professional who is trained in diagnosing or classifying eating disorders in patients. Because different clinicians might be influenced by their training or limited (or different) experiences, imprecise and/or subjective diagnoses or classifications of eating disorders may result. Further compounding this issue is that even if an experienced clinician performs such diagnoses or classifications of eating disorders in patients, such a clinician is only one person and can only interview and diagnose a limited number of subjects or patients within a set time.

Hence, there is a need for more robust and scalable solutions for implementing eating disorder diagnosis, and, more particularly, to methods, systems, and apparatuses for implementing eating disorder diagnosis based on analysis of patient responses to a set of questions having closed-ended answer options.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. In some instances, a sub-label is associated with a reference numeral to denote one of multiple similar components. When reference is made to a reference numeral without specification to an existing sub-label, it is intended to refer to all such multiple similar components.

FIG. 1 is a schematic diagram illustrating a system for implementing eating disorder diagnosis, in accordance with various embodiments.

FIGS. 2A and 2B are graphical diagrams illustrating non-limiting examples of estimated probabilities of eating disorders for subjects diagnosed as having eating disorders and for subjects diagnosed as having no eating disorders, respectively, in response to the implemented eating disorder diagnosis, in accordance with various embodiments.

FIG. 2C is a graphical diagram illustrating a non-limiting example of misclassification of eating disorders versus predicted eating disorders, in accordance with various embodiments.

FIGS. 2D and 2E illustrate non-limiting examples of tables listing p-values for corresponding variables and listing classification functions with item weights for each eating disorder, respectively, in accordance with various embodiments.

FIGS. 2F and 2G illustrate non-limiting examples of tables listing the percentage of correct diagnoses of eating disorders for a group of individuals and listing percentage of correct diagnosis of eating disorder for particular patients, in accordance with various embodiments.

FIGS. 3A and 3B are schematic diagrams illustrating another non-limiting example of a user device that presents an exemplary graphical user interface for displaying diagnosis of eating disorders, in accordance with various embodiments.

FIGS. 4A-4C are flow diagrams illustrating a method for implementing eating disorder diagnosis, in accordance with various embodiments.

FIG. 5 is a block diagram illustrating an exemplary computer or system hardware architecture, in accordance with various embodiments.

FIG. 6 is a block diagram illustrating a networked system of computers, computing systems, or system hardware architecture, which can be used in accordance with various embodiments.

### DETAILED DESCRIPTION OF CERTAIN EMBODIMENTS

Overview

Various embodiments provide tools and techniques for implementing eating disorder diagnosis, and, more particularly, to methods, systems, and apparatuses for implementing eating disorder diagnosis based on analysis of patient responses to a set of questions having closed-ended answer options.

In various embodiments, a computing system might receive, from a first patient, a first set of patient responses to a set of questions each having closed-ended answer options. Each question might be dichotomized such that a first set of answer options among its closed-ended answer options are assigned a first score while a second set of answer options among its closed-ended answer options are assigned a second score.

In some embodiments, the set of questions might include, without limitation, a first category of questions, a second category of questions, and a third category of questions, or the like. The first category of questions might comprise questions regarding conditions including, but not limited to, at least one of body-mass index (“BMI”), weight loss during the previous year, or self-induced vomiting, and/or the like. The second category of questions might comprise questions regarding behavior including, without limitation, at least one of eating patterns, dieting, weighing one's self, isolation from friends and family, or avoiding activities, and/or the like. The third category of questions might comprise questions regarding thoughts including, but not limited to, at least one of being afraid of losing control over eating, thoughts about food, believing one's self to be fat when others call one too thin, or reaction to weight gain, and/or the like. The first set of weighted values might be differently defined based on differences among the first category of questions, the second category of questions, and the third category of questions. The computing system might determine, for each dichotomized question among the set of dichotomized questions, whether a corresponding patient response among the first set of patient responses corresponds to the first set of answer options for that dichotomized question or corresponds to the second set of answer options for that dichotomized question, wherein the first set of answer options for the set of dichotomized questions is indicative of likelihood of patients selecting such answer options having an eating disorder, and wherein the second set of answer options for the set of dichotomized questions is indicative of likelihood of patients selecting such answer options not having an eating disorder.

The computing system might determine a diagnosis of whether or not the first patient has an eating disorder, based at least in part on logistic regression analysis of one or more of the patient responses among the first set of patient responses corresponding to the first set of answer options for the set of dichotomized questions or the patient responses among the first set of patient responses corresponding to the second set of answer options for the set of dichotomized questions. Based on a determination that the first patient likely has an eating disorder, the computing system might perform one or more of the following: (a) identify a first set of weighted values each corresponding to each of the received first set of patient responses from the first patient, and calculate a first probability of diagnosis of a first eating disorder, based at least in part on modification of the received first set of patient responses by multiplication with the first set of weighted values and by subsequent addition of a first constant value associated with diagnosis of the first eating disorder; (b) identify a second set of weighted values each corresponding to each of the received first set of patient responses from the first patient, and calculate a second probability of diagnosis of a second eating disorder, based at least in part on modification of the received second set of patient responses by multiplication with the second set of weighted values and by subsequent addition of a second constant value associated with diagnosis of the second eating disorder; (c) identify a third set of weighted values each corresponding to each of the received first set of patient responses from the first patient, and calculate a third probability of diagnosis of a third eating disorder, based at least in part on modification of the received third set of patient responses by multiplication with the third set of weighted values and by subsequent addition of a third constant value associated with diagnosis of the third eating disorder; (d) identify a fourth set of weighted values each corresponding to each of the received first set of patient responses from the first patient, and calculate a fourth probability of diagnosis of a fourth eating disorder, based at least in part on modification of the received fourth set of patient responses by multiplication with the fourth set of weighted values and by subsequent addition of a fourth constant value associated with diagnosis of the fourth eating disorder; and/or (e) identify a fifth set of weighted values each corresponding to each of the received first set of patient responses from the first patient, and might calculate a fifth probability of diagnosis of a fifth eating disorder, based at least in part on modification of the received fifth set of patient responses by multiplication with the fifth set of weighted values and by subsequent addition of a fifth constant value associated with diagnosis of the fifth eating disorder; and/or the like. In some embodiments, the first eating disorder, the second eating disorder, the third eating disorder, the fourth eating disorder, and the fifth eating disorder might each comprise one of anorexia nervosa (“AN”), bulimia nervosa (“BN”), binge-eating disorder (“BED”), obesity (“OB”), or other specified feeding or eating disorder (“OSFED”), and/or the like.

The computing system might identify which eating disorder the first patient is likely to have, based at least in part on the determined diagnosis and based at least in part on the calculated first through fifth probabilities. According to some embodiments, the computing system might identify suggested therapy techniques for the identified eating disorder. Optionally, the computing system might receive diagnosis of the first patient performed by a clinician or healthcare professional. In such cases, the computing system might compare the identified eating disorder of the first patient with the received diagnosis of the first patient performed by the clinician or healthcare professional to determine whether the identified eating disorder matches the received diagnosis. In some instances, the computing system might display, on a display device, the identified eating disorder and/or the received diagnosis of the first patient performed by the clinician or healthcare professional. In some cases, displaying the identified eating disorder might comprise displaying, with the computing system, the identified eating disorder in a software application (“app”) running on the display device. In some embodiments, the computing system might send a message to a user device(s) associated with the clinician or healthcare professional, the message comprising the identified eating disorder associated with the first patient. In some instances, the message might further comprise the identified suggested therapy techniques associated with the identified eating disorder associated with the first patient.

According to some embodiments, the computing system might receive a plurality of patient responses associated with a plurality of patients, an identified eating disorder associated with each patient among the plurality of patients, and a diagnosis of each patient among the plurality of patients performed by one or more clinicians or healthcare professionals. The computing system might compare the identified eating disorder associated with each patient among the plurality of patients with the received diagnosis of each patient performed by the one or more clinicians or healthcare professionals to determine whether the identified eating disorder matches the received diagnosis. Based on the comparison, the computing system might analyze the plurality of sets of patient responses associated with the plurality of patients, the identified eating disorder associated with each patient among the plurality of patients, the diagnosis of each patient among the plurality of patients performed by the one or more clinicians or healthcare professionals, and one or more of the first through fifth set of weighted values to determine whether one or more of the first set of weighted values, the second set of weighted values, the third set of weighted values, the fourth set of weighted values, the fifth set of weighted values, the first constant value associated with diagnosis of the first eating disorder, the second constant value associated with diagnosis of the second eating disorder, the third constant value associated with diagnosis of the third eating disorder, the fourth constant value associated with diagnosis of the fourth eating disorder, or the fifth constant value associated with diagnosis of the fifth eating disorder are optimal or should be updated or modified, and/or the like.

The computing system might modify one or more of the first set of weighted values, the second set of weighted values, the third set of weighted values, the fourth set of weighted values, the fifth set of weighted values, the first constant value associated with diagnosis of the first eating disorder, the second constant value associated with diagnosis of the second eating disorder, the third constant value associated with diagnosis of the third eating disorder, the fourth constant value associated with diagnosis of the fourth eating disorder, or the fifth constant value associated with diagnosis of the fifth eating disorder, based at least in part on the comparison of the identified eating disorder associated with each patient among the plurality of patients (including the first patient) with the received diagnosis of each patient performed by the one or more clinicians or healthcare professionals, and based at least in part on the received plurality of patient responses associated with the plurality of patients.

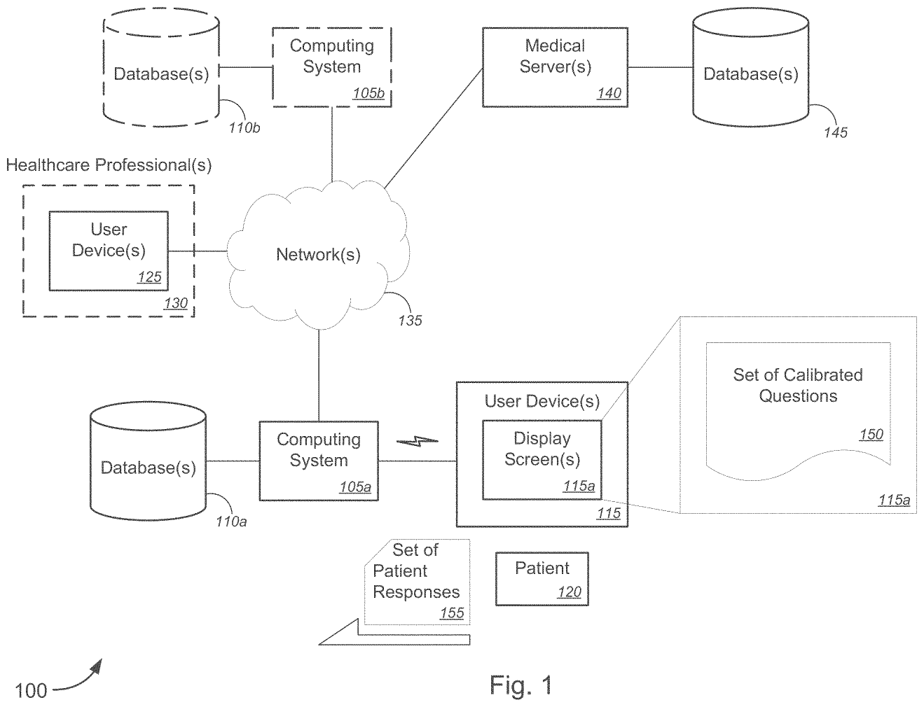

The various embodiments provide for techniques and systems for implementing eating disorder diagnosis based on analysis of patient responses to a set of questions having closed-ended answer options. Such eating disorder diagnosis, as described in greater detail below with respect to the figures, result in optimized, relatively quick, accurate, and objective diagnosis or classification of patient eating disorders (as opposed to imprecise and subjective diagnosis or classification by differing clinicians or healthcare providers, who may or may not be influenced by their training or limited experiences), which would allow for more targeted or tailored treatment of such eating disorders for each particular patient, and/or the like. Further, because a nurse, social worker, or receptionist can hand out user devices (e.g., tablet computers, etc.) that a subject or patient can use to complete the answers to the questions and that can either perform the processes of the computing system as described with respect to FIG. 1 or the like, or that can send the patient responses to such computing system, the various embodiments can free doctors to oversee the diagnosis of patients whose identified or diagnosed eating disorders are ambiguous or inconclusive (e.g., as in the case with patients having multiple eating disorders, or the like).

These and other aspects of the eating disorder diagnosis are described in greater detail with respect to the figures.

The following detailed description illustrates a few exemplary embodiments in further detail to enable one of skill in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art, however, that other embodiments of the present invention may be practiced without some of these specific details. In other instances, certain structures and devices are shown in block diagram form. Several embodiments are described herein, and while various features are ascribed to different embodiments, it should be appreciated that the features described with respect to one embodiment may be incorporated with other embodiments as well. By the same token, however, no single feature or features of any described embodiment should be considered essential to every embodiment of the invention, as other embodiments of the invention may omit such features.

Unless otherwise indicated, all numbers used herein to express quantities, dimensions, and so forth used should be understood as being modified in all instances by the term “about.” In this application, the use of the singular includes the plural unless specifically stated otherwise, and use of the terms “and” and “or” means “and/or” unless otherwise indicated. Moreover, the use of the term “including,” as well as other forms, such as “includes” and “included,” should be considered non-exclusive. Also, terms such as “element” or “component” encompass both elements and components comprising one unit and elements and components that comprise more than one unit, unless specifically stated otherwise.

Various embodiments described herein, while embodying (in some cases) software products, computer-performed methods, and/or computer systems, represent tangible, concrete improvements to existing technological areas, including, without limitation, patient diagnosis technologies, patient diagnosis technologies, patient eating disorder diagnosis technologies, patient eating disorder treatment technologies, and/or the like. In other aspects, certain embodiments, can improve the functioning of user equipment or systems themselves (e.g., patient diagnosis systems, patient diagnosis systems, patient eating disorder diagnosis systems, patient eating disorder treatment systems, etc.), for example, by receiving, with a computing system and from a first patient, a first set of patient responses to a set of questions each having closed-ended answer options, wherein each question is dichotomized such that a first set of answer options among its closed-ended answer options are assigned a first score while a second set of answer options among its closed-ended answer options are assigned a second score; determining, with the computing system and for each dichotomized question among the set of dichotomized questions, whether a corresponding patient response among the first set of patient responses corresponds to the first set of answer options for that dichotomized question or corresponds to the second set of answer options for that dichotomized question, wherein the first set of answer options for the set of dichotomized questions is indicative of likelihood of patients selecting such answer options having an eating disorder, and wherein the second set of answer options for the set of dichotomized questions is indicative of likelihood of patients selecting such answer options not having an eating disorder; determining, with the computing system, a diagnosis of whether or not the first patient has an eating disorder, based at least in part on logistic regression analysis of one or more of the patient responses among the first set of patient responses corresponding to the first set of answer options for the set of dichotomized questions or the patient responses among the first set of patient responses corresponding to the second set of answer options for the set of dichotomized questions; and based on a determination that the first patient likely has an eating disorder, performing one or more of the following: identifying, with the computing system, one or more of a first through fifth set of weighted values each corresponding to each of the received first set of patient responses from the first patient; calculating, with the computing system, a corresponding one or more of a first through fifth probability of diagnosis of a first through fifth eating disorder, each based at least in part on modification of the received first set of patient responses by multiplication with the corresponding one of the first through fifth set of weighted values and by subsequent addition of a corresponding one of the first through fifth constant value associated with diagnosis of the corresponding one of the first through fifth eating disorder (where the first through fifth eating disorder each comprises one of anorexia nervosa (“AN”), bulimia nervosa (“BN”), binge-eating disorder (“BED”), obesity (“OB”), or other specified feeding or eating disorder (“OSFED”)); identifying, with the computing system, which eating disorder the first patient is likely to have, based at least in part on the determined diagnosis and based at least in part on the calculated first through fifth probabilities; and/or the like.

In particular, to the extent any abstract concepts are present in the various embodiments, those concepts can be implemented as described herein by devices, software, systems, and methods that involve specific novel functionality (e.g., steps or operations), such as, analyzing patient responses to a set of questions each having closed-ended answer options, wherein each question is dichotomized such that a first set of answer options among its closed-ended answer options are assigned a first score while a second set of answer options among its closed-ended answer options are assigned a second score, wherein the first set of answer options for the set of dichotomized questions is indicative of likelihood of patients selecting such answer options having an eating disorder, and wherein the second set of answer options for the set of dichotomized questions is indicative of likelihood of patients selecting such answer options not having an eating disorder; determining a diagnosis of whether or not the patient has an eating disorder, based at least in part on logistic regression analysis of one or more of the patient responses among the first set of patient responses corresponding to the first set of answer options for the set of dichotomized questions or the patient responses among the first set of patient responses corresponding to the second set of answer options for the set of dichotomized questions; and based on a determination that the first patient likely has an eating disorder, performing one or more of the following: identifying weighted values corresponding to each of the received set of patient responses and calculating for each of one or more eating disorders (e.g., AN, BN, BED, OB, and OSFED) a probability of diagnosis of such eating disorder, based at least in part on modification of the received first set of patient responses by multiplication with said set of weighted values and by subsequent addition of a corresponding constant value associated with diagnosis of that eating disorder; identifying which eating disorder the patient is likely to have, based at least in part on the determined diagnosis and based at least in part on the calculated probabilities; and/or the like, to name a few examples, that extend beyond mere conventional computer processing operations. These functionalities can produce tangible results outside of the implementing computer system, including, merely by way of example, optimized, relatively quick, accurate, and objective diagnosis or classification of patient eating disorders (as opposed to imprecise and subjective diagnosis or classification by differing clinicians or healthcare providers, who may or may not be influenced by their training or limited experiences), which would allow for more targeted or tailored treatment of such eating disorders for each particular patient, and/or the like, at least some of which may be observed or measured by patients or subjects, and/or healthcare providers.

In an aspect, a method might comprise receiving, with a computing system and from a first patient, a first set of patient responses to a set of questions each having closed-ended answer options, wherein each question is dichotomized such that a first set of answer options among its closed-ended answer options are assigned a first score while a second set of answer options among its closed-ended answer options are assigned a second score; and determining, with the computing system and for each dichotomized question among the set of dichotomized questions, whether a corresponding patient response among the first set of patient responses corresponds to the first set of answer options for that dichotomized question or corresponds to the second set of answer options for that dichotomized question, wherein the first set of answer options for the set of dichotomized questions is indicative of likelihood of patients selecting such answer options having an eating disorder, and wherein the second set of answer options for the set of dichotomized questions is indicative of likelihood of patients selecting such answer options not having an eating disorder.

The method might further comprise determining, with the computing system, a diagnosis of whether or not the first patient has an eating disorder, based at least in part on logistic regression analysis of one or more of the patient responses among the first set of patient responses corresponding to the first set of answer options for the set of dichotomized questions or the patient responses among the first set of patient responses corresponding to the second set of answer options for the set of dichotomized questions; and based on a determination that the first patient likely has an eating disorder, performing one or more of the following: identifying, with the computing system, a first set of weighted values each corresponding to each of the received first set of patient responses from the first patient; calculating, with the computing system, a first probability of diagnosis of anorexia nervosa (“AN”), based at least in part on modification of the received first set of patient responses by multiplication with the first set of weighted values and by subsequent addition of a first constant value associated with diagnosis of AN; identifying, with the computing system, a second set of weighted values each corresponding to each of the received first set of patient responses from the first patient; calculating, with the computing system, a second probability of diagnosis of bulimia nervosa (“BN”), based at least in part on modification of the received second set of patient responses by multiplication with the second set of weighted values and by subsequent addition of a second constant value associated with diagnosis of BN; identifying, with the computing system, a third set of weighted values each corresponding to each of the received first set of patient responses from the first patient; calculating, with the computing system, a third probability of diagnosis of binge-eating disorder (“BED”), based at least in part on modification of the received third set of patient responses by multiplication with the third set of weighted values and by subsequent addition of a third constant value associated with diagnosis of BED; identifying, with the computing system, a fourth set of weighted values each corresponding to each of the received first set of patient responses from the first patient; calculating, with the computing system, a fourth probability of diagnosis of obesity (“OB”), based at least in part on modification of the received fourth set of patient responses by multiplication with the fourth set of weighted values and by subsequent addition of a fourth constant value associated with diagnosis of OB; identifying, with the computing system, a fifth set of weighted values each corresponding to each of the received first set of patient responses from the first patient; calculating, with the computing system, a fifth probability of diagnosis of other specified feeding or eating disorder (“OSFED”), based at least in part on modification of the received fifth set of patient responses by multiplication with the fifth set of weighted values and by subsequent addition of a fifth constant value associated with diagnosis of OSFED.

The method might also comprise identifying, with the computing system, which eating disorder the first patient is likely to have, based at least in part on the determined diagnosis and based at least in part on the calculated first through fifth probabilities. The method might comprise identifying, with the computing system, suggested therapy techniques for the identified eating disorder.

The method might further comprise modifying, with the computing system, one or more of the first set of weighted values, the second set of weighted values, the third set of weighted values, the fourth set of weighted values, the fifth set of weighted values, the first constant value associated with diagnosis of AN, the second constant value associated with diagnosis of BN, the third constant value associated with diagnosis of BED, the fourth constant value associated with diagnosis of OB, or the fifth constant value associated with diagnosis of OSFED, based at least in part on one or more of the received first set of patient responses and/or a plurality of patient responses associated with a plurality of patients.

In another aspect, a method might comprise receiving, with a computing system and from a first patient, a first set of patient responses to a set of questions each having closed-ended answer options; autonomously determining, with the computing system, a diagnosis of whether or not the first patient has an eating disorder, based at least in part on logistic regression analysis of the first set of patient responses; and based on a determination that the first patient likely has an eating disorder, performing one or more of the following: autonomously identifying, with the computing system, a first set of weighted values each corresponding to each of the received first set of patient responses from the first patient; autonomously calculating, with the computing system, a first probability of diagnosis of a first eating disorder, based at least in part on modification of the received first set of patient responses by multiplication with the first set of weighted values and by subsequent addition of a first constant value associated with diagnosis of the first eating disorder; autonomously identifying, with the computing system, a second set of weighted values each corresponding to each of the received first set of patient responses from the first patient; and autonomously calculating, with the computing system, a second probability of diagnosis of a second eating disorder, based at least in part on modification of the received second set of patient responses by multiplication with the second set of weighted values and by subsequent addition of a second constant value associated with diagnosis of the second eating disorder.

The method might further comprise autonomously identifying, with the computing system, which eating disorder the first patient is likely to have, based at least in part on the determined diagnosis and based at least in part on the calculated first and second probabilities; and displaying, with the computing system and on a display device, the identified eating disorder.

In some embodiments, each question might be dichotomized such that a first set of answer options among its closed-ended answer options are assigned a first score while a second set of answer options among its closed-ended answer options are assigned a second score, wherein the first score is indicative of likelihood of patients selecting such answer options having an eating disorder, and wherein the second score is indicative of likelihood of patients selecting such answer options not having an eating disorder. In such cases, the method might further comprise autonomously determining, with the computing system and for each dichotomized question among the set of dichotomized questions, whether a corresponding patient response among the first set of patient responses corresponds to the first set of answer options for that dichotomized question or corresponds to the second set of answer options for that dichotomized question. In some cases, autonomously determining the diagnosis of whether or not the first patient has an eating disorder might be further based at least in part on logistic regression analysis of one or more of the patient responses among the first set of patient responses corresponding to the first set of answer options that are assigned the first score or the patient responses among the first set of patient responses corresponding to the second set of answer options that are assigned the second score.

According to some embodiments, the method might further comprise, based on the determination that the first patient likely has an eating disorder: autonomously identifying, with the computing system, a third set of weighted values each corresponding to each of the received first set of patient responses from the first patient; autonomously calculating, with the computing system, a third probability of diagnosis of a third eating disorder, based at least in part on modification of the received third set of patient responses by multiplication with the third set of weighted values and by subsequent addition of a third constant value associated with diagnosis of the third eating disorder; autonomously identifying, with the computing system, a fourth set of weighted values each corresponding to each of the received first set of patient responses from the first patient; autonomously calculating, with the computing system, a fourth probability of diagnosis of a fourth eating disorder, based at least in part on modification of the received fourth set of patient responses by multiplication with the fourth set of weighted values and by subsequent addition of a fourth constant value associated with diagnosis of the fourth eating disorder; autonomously identifying, with the computing system, a fifth set of weighted values each corresponding to each of the received first set of patient responses from the first patient; and autonomously calculating, with the computing system, a fifth probability of diagnosis of a fifth eating disorder, based at least in part on modification of the received fifth set of patient responses by multiplication with the fifth set of weighted values and by subsequent addition of a fifth constant value associated with diagnosis of the fifth eating disorder. In such cases, autonomously identifying which eating disorder the first patient is likely to have might comprise autonomously identifying, with the computing system, which eating disorder the first patient is likely to have, based at least in part on the determined diagnosis and based at least in part on the calculated first through fifth probabilities.

In some cases, the first eating disorder, the second eating disorder, the third eating disorder, the fourth eating disorder, and the fifth eating disorder might each comprise one of anorexia nervosa (“AN”), bulimia nervosa (“BN”), binge-eating disorder (“BED”), obesity (“OB”), or other specified feeding or eating disorder (“OSFED”), and/or the like. In some instances, the first patient might be among a plurality of patients, where the method might further comprise receiving, with the computing system, a plurality of sets of patient responses associated with the plurality of patients, an identified eating disorder associated with each patient among the plurality of patients, and a diagnosis of each patient among the plurality of patients performed by one or more clinicians, wherein the plurality of patient response comprises the first set of patient responses associated with the first patient; autonomously comparing, with the computing system, the identified eating disorder associated with each patient among the plurality of patients with the received diagnosis of each patient performed by the one or more clinicians to determine whether the identified eating disorder matches the received diagnosis; based on the comparison, autonomously analyzing, with the computing system, the plurality of sets of patient responses associated with the plurality of patients, the identified eating disorder associated with each patient among the plurality of patients, the diagnosis of each patient among the plurality of patients performed by the one or more clinicians, and the first through fifth set of weighted values to determine whether one or more of the first set of weighted values, the second set of weighted values, the third set of weighted values, the fourth set of weighted values, the fifth set of weighted values, the first constant value associated with diagnosis of the first eating disorder, the second constant value associated with diagnosis of the second eating disorder, the third constant value associated with diagnosis of the third eating disorder, the fourth constant value associated with diagnosis of the fourth eating disorder, or the fifth constant value associated with diagnosis of the fifth eating disorder are optimal or should be updated or modified; and autonomously modifying, with the computing system, one or more of the first set of weighted values, the second set of weighted values, the third set of weighted values, the fourth set of weighted values, the fifth set of weighted values, the first constant value associated with diagnosis of the first eating disorder, the second constant value associated with diagnosis of the second eating disorder, the third constant value associated with diagnosis of the third eating disorder, the fourth constant value associated with diagnosis of the fourth eating disorder, or the fifth constant value associated with diagnosis of the fifth eating disorder, based at least in part on one or more of the comparison of the identified eating disorder associated with each patient among the plurality of patients with the received diagnosis of each patient performed by the one or more clinicians, the received plurality of sets of patient responses associated with the plurality of patients, or the analysis.

In some embodiments, at least one of autonomously identifying each of the first through fifth set of weighted values, autonomously calculating the first through fifth probability of diagnosis, autonomously identifying which eating disorder the first patient is likely to have, autonomously comparing the identified eating disorder associated with each patient among the plurality of patients with the received diagnosis of each patient performed by the one or more clinicians, or autonomously modifying the one or more of the first set of weighted values, the second set of weighted values, the third set of weighted values, the fourth set of weighted values, the fifth set of weighted values, the first constant value associated with diagnosis of the first eating disorder, the second constant value associated with diagnosis of the second eating disorder, the third constant value associated with diagnosis of the third eating disorder, the fourth constant value associated with diagnosis of the fourth eating disorder, or the fifth constant value associated with diagnosis of the fifth eating disorder might be performed in near-real-time.

Merely by way of example, in some cases, the display device might comprise one of a tablet computer, a smart phone, a mobile phone, a laptop computer, a desktop computer, or a monitor, and/or the like. In some instances, displaying the identified eating disorder might comprise displaying, with the computing system, the identified eating disorder in a software application (“app”) running on the display device. In some embodiments, receiving the first set of patient responses might comprise receiving, with the computing system and from a user device that receives input from the first patient, the first set of patient responses to the set of questions.

According to some embodiments, the method might further comprise sending, with the computing system, a message to a user device associated with a medical practitioner, the message comprising the identified eating disorder associated with the first patient. In some cases, the medical practitioner might comprise one of a general medical practitioner, a primary care physician, a psychiatrist, a clinician, or a nurse, and/or the like. In some instances, the user device associated with the medical practitioner might comprise one of a tablet computer, a smart phone, a mobile phone, a laptop computer, or a desktop computer, and/or the like. In some cases, the message might further comprise suggested therapy techniques associated with the identified eating disorder associated with the first patient.

Merely by way of example, in some cases, the set of questions might comprise a first category of questions, a second category of questions, and a third category of questions. In some cases, the first category of questions might comprise questions regarding conditions including at least one of body-mass index (“BMI”), weight loss during the previous year, or self-induced vomiting, and/or the like. In some instances, the second category of questions might comprise questions regarding behavior including at least one of eating patterns, dieting, weighing one's self, isolation from friends and family, or avoiding activities, and/or the like. In some cases, the third category of questions might comprise questions regarding thoughts including at least one of being afraid of losing control over eating, thoughts about food, believing one's self to be fat when others call one too thin, or reaction to weight gain, and/or the like. In some instances, the first set of weighted values might be differently defined based on differences among the first category of questions, the second category of questions, and the third category of questions.

In yet another aspect, a system might comprise a computing system, which might comprise at least one first processor and a first non-transitory computer readable medium communicatively coupled to the at least one first processor. The first non-transitory computer readable medium might have stored thereon computer software comprising a first set of instructions that, when executed by the at least one first processor, causes the computing system to: receive, from a first patient, a first set of patient responses to a set of questions each having closed-ended answer options; autonomously determine a diagnosis of whether or not the first patient has an eating disorder, based at least in part on logistic regression analysis of the first set of patient responses; and based on a determination that the first patient likely has an eating disorder, perform one or more of the following: autonomously identify a first set of weighted values each corresponding to each of the received first set of patient responses from the first patient; autonomously calculate a first probability of diagnosis of a first eating disorder, based at least in part on modification of the received first set of patient responses by multiplication with the first set of weighted values and by subsequent addition of a first constant value associated with diagnosis of the first eating disorder; autonomously identify a second set of weighted values each corresponding to each of the received first set of patient responses from the first patient; autonomously calculate a second probability of diagnosis of a second eating disorder, based at least in part on modification of the received second set of patient responses by multiplication with the second set of weighted values and by subsequent addition of a second constant value associated with diagnosis of the second eating disorder; autonomously identify which eating disorder the first patient is likely to have, based at least in part on the determined diagnosis and based at least in part on the calculated first and second probabilities; and display, on a display device, the identified eating disorder.

In some embodiments, each question might be dichotomized such that a first set of answer options among its closed-ended answer options are assigned a first score while a second set of answer options among its closed-ended answer options are assigned a second score, wherein the first score is indicative of likelihood of patients selecting such answer options having an eating disorder, and wherein the second score is indicative of likelihood of patients selecting such answer options not having an eating disorder, wherein the first set of instructions, when executed by the at least one first processor, might further cause the computing system to: autonomously determine, for each dichotomized question among the set of dichotomized questions, whether a corresponding patient response among the first set of patient responses corresponds to the first set of answer options for that dichotomized question or corresponds to the second set of answer options for that dichotomized question. In some cases, autonomously determining the diagnosis of whether or not the first patient has an eating disorder might be further based at least in part on logistic regression analysis of one or more of the patient responses among the first set of patient responses corresponding to the first set of answer options that are assigned the first score or the patient responses among the first set of patient responses corresponding to the second set of answer options that are assigned the second score.

According to some embodiments, based on the determination that the first patient likely has an eating disorder, the first set of instructions, when executed by the at least one first processor, might further cause the computing system to: autonomously identify a third set of weighted values each corresponding to each of the received first set of patient responses from the first patient; autonomously calculate a third probability of diagnosis of a third eating disorder, based at least in part on modification of the received third set of patient responses by multiplication with the third set of weighted values and by subsequent addition of a third constant value associated with diagnosis of the third eating disorder; autonomously identify a fourth set of weighted values each corresponding to each of the received first set of patient responses from the first patient; autonomously calculate a fourth probability of diagnosis of a fourth eating disorder, based at least in part on modification of the received fourth set of patient responses by multiplication with the fourth set of weighted values and by subsequent addition of a fourth constant value associated with diagnosis of the fourth eating disorder; and autonomously identify a fifth set of weighted values each corresponding to each of the received first set of patient responses from the first patient; autonomously calculate a fifth probability of diagnosis of a fifth eating disorder, based at least in part on modification of the received fifth set of patient responses by multiplication with the fifth set of weighted values and by subsequent addition of a fifth constant value associated with diagnosis of the fifth eating disorder. In some instances, autonomously identifying which eating disorder the first patient is likely to have might comprise autonomously identifying which eating disorder the first patient is likely to have, based at least in part on the determined diagnosis and based at least in part on the calculated first through fifth probabilities.

In some embodiments, the first eating disorder, the second eating disorder, the third eating disorder, the fourth eating disorder, and the fifth eating disorder might each comprise one of anorexia nervosa (“AN”), bulimia nervosa (“BN”), binge-eating disorder (“BED”), obesity (“OB”), or other specified feeding or eating disorder (“OSFED”), and/or the like.

According to some embodiments, the first patient might be among a plurality of patients, where the first set of instructions, when executed by the at least one first processor, might further cause the computing system to: receive a plurality of sets of patient responses associated with the plurality of patients, an identified eating disorder associated with each patient among the plurality of patients, and a diagnosis of each patient among the plurality of patients performed by one or more clinicians, wherein the plurality of patient response comprises the first set of patient responses associated with the first patient; autonomously compare the identified eating disorder associated with each patient among the plurality of patients with the received diagnosis of each patient performed by the one or more clinicians to determine whether the identified eating disorder matches the received diagnosis; based on the comparison, autonomously analyze the plurality of sets of patient responses associated with the plurality of patients, an identified eating disorder associated with each patient among the plurality of patients, a diagnosis of each patient among the plurality of patients performed by the one or more clinicians, and the first through fifth set of weighted values to determine whether one or more of the first set of weighted values, the second set of weighted values, the third set of weighted values, the fourth set of weighted values, the fifth set of weighted values, the first constant value associated with diagnosis of the first eating disorder, the second constant value associated with diagnosis of the second eating disorder, the third constant value associated with diagnosis of the third eating disorder, the fourth constant value associated with diagnosis of the fourth eating disorder, or the fifth constant value associated with diagnosis of the fifth eating disorder are optimal or should be updated or modified; and autonomously modify one or more of the first set of weighted values, the second set of weighted values, the third set of weighted values, the fourth set of weighted values, the fifth set of weighted values, the first constant value associated with diagnosis of the first eating disorder, the second constant value associated with diagnosis of the second eating disorder, the third constant value associated with diagnosis of the third eating disorder, the fourth constant value associated with diagnosis of the fourth eating disorder, or the fifth constant value associated with diagnosis of the fifth eating disorder, based at least in part on one or more of the comparison of the identified eating disorder associated with each patient among the plurality of patients with the received diagnosis of each patient performed by the one or more clinicians, the received plurality of sets of patient responses associated with the plurality of patients, or the analysis.

In still another aspect, a method might comprise receiving, with a computing system, a plurality of sets of patient responses to a set of questions each having closed-ended answer options, the plurality of sets of patient responses being associated with a plurality of patients, an identified eating disorder associated with each patient among the plurality of patients, and a diagnosis of each patient among the plurality of patients performed by one or more clinicians; autonomously comparing, with the computing system, the identified eating disorder associated with each patient among the plurality of patients with the received diagnosis of each patient performed by the one or more clinicians to determine whether the identified eating disorder matches the received diagnosis; and autonomously modifying, with the computing system, one or more of a first set of weighted values, a second set of weighted values, a third set of weighted values, a fourth set of weighted values, a fifth set of weighted values, a first constant value associated with diagnosis of a first eating disorder, a second constant value associated with diagnosis of a second eating disorder, a third constant value associated with diagnosis of a third eating disorder, a fourth constant value associated with diagnosis of a fourth eating disorder, or a fifth constant value associated with diagnosis of a fifth eating disorder, based at least in part on one or more of the comparison of the identified eating disorder associated with each patient among the plurality of patients with the received diagnosis of each patient performed by the one or more clinicians or the received plurality of patient responses associated with the plurality of patients.

In some embodiments, the method might further comprise, based on the comparison, autonomously analyzing, with the computing system, the plurality of sets of patient responses associated with the plurality of patients, the identified eating disorder associated with each patient among the plurality of patients, the diagnosis of each patient among the plurality of patients performed by the one or more clinicians, and the first through fifth set of weighted values to determine whether one or more of the first set of weighted values, the second set of weighted values, the third set of weighted values, the fourth set of weighted values, the fifth set of weighted values, the first constant value associated with diagnosis of the first eating disorder, the second constant value associated with diagnosis of the second eating disorder, the third constant value associated with diagnosis of the third eating disorder, the fourth constant value associated with diagnosis of the fourth eating disorder, or the fifth constant value associated with diagnosis of the fifth eating disorder are optimal or should be updated or modified.

According to some embodiments, the first set of weighted values, the second set of weighted values, the third set of weighted values, the fourth set of weighted values, and the fifth set of weighted values might each correspond to each of the received plurality of sets of patient responses. In some cases, the first eating disorder, the second eating disorder, the third eating disorder, the fourth eating disorder, and the fifth eating disorder might each comprise one of anorexia nervosa (“AN”), bulimia nervosa (“BN”), binge-eating disorder (“BED”), obesity (“OB”), or other specified feeding or eating disorder (“OSFED”), and/or the like.

In some embodiments, at least one of autonomously comparing the identified eating disorder associated with each patient among the plurality of patients with the received diagnosis of each patient performed by the one or more clinicians or autonomously modifying the one or more of the first set of weighted values, the second set of weighted values, the third set of weighted values, the fourth set of weighted values, the fifth set of weighted values, the first constant value associated with diagnosis of the first eating disorder, the second constant value associated with diagnosis of the second eating disorder, the third constant value associated with diagnosis of the third eating disorder, the fourth constant value associated with diagnosis of the fourth eating disorder, or the fifth constant value associated with diagnosis of the fifth eating disorder might be performed in near-real-time.

According to some embodiments, the set of questions might comprise a first category of questions, a second category of questions, and a third category of questions. In some cases, the first category of questions might comprise questions regarding conditions including at least one of body-mass index (“BMI”), weight loss during the previous year, or self-induced vomiting, and/or the like. In some instances, the second category of questions might comprise questions regarding behavior including at least one of eating patterns, dieting, weighing one's self, isolation from friends and family, or avoiding activities, and/or the like. In some cases, the third category of questions might comprise questions regarding thoughts including at least one of being afraid of losing control over eating, thoughts about food, believing one's self to be fat when others call one too thin, or reaction to weight gain, and/or the like. In some instances, the first set of weighted values might be differently defined based on differences among the first category of questions, the second category of questions, and the third category of questions.

In an aspect, a system might comprise a computing system, which might comprise at least one first processor and a first non-transitory computer readable medium communicatively coupled to the at least one first processor. The first non-transitory computer readable medium might have stored thereon computer software comprising a first set of instructions that, when executed by the at least one first processor, causes the computing system to: receive a plurality of sets of patient responses to a set of questions each having closed-ended answer options, the plurality of sets of patient responses being associated with a plurality of patients, an identified eating disorder associated with each patient among the plurality of patients, and a diagnosis of each patient among the plurality of patients performed by one or more clinicians; autonomously compare the identified eating disorder associated with each patient among the plurality of patients with the received diagnosis of each patient performed by the one or more clinicians to determine whether the identified eating disorder matches the received diagnosis; and autonomously modify one or more of a first set of weighted values, a second set of weighted values, a third set of weighted values, a fourth set of weighted values, a fifth set of weighted values, a first constant value associated with diagnosis of a first eating disorder, a second constant value associated with diagnosis of a second eating disorder, a third constant value associated with diagnosis of a third eating disorder, a fourth constant value associated with diagnosis of a fourth eating disorder, or a fifth constant value associated with diagnosis of a fifth eating disorder, based at least in part on one or more of the comparison of the identified eating disorder associated with each patient among the plurality of patients with the received diagnosis of each patient performed by the one or more clinicians or the received plurality of patient responses associated with the plurality of patients.

In some embodiments, the first set of weighted values, the second set of weighted values, the third set of weighted values, the fourth set of weighted values, and the fifth set of weighted values might each correspond to each of the received plurality of sets of patient responses. In some instances, the first eating disorder, the second eating disorder, the third eating disorder, the fourth eating disorder, and the fifth eating disorder might each comprise one of anorexia nervosa (“AN”), bulimia nervosa (“BN”), binge-eating disorder (“BED”), obesity (“OB”), or other specified feeding or eating disorder (“OSFED”), and/or the like.

According to some embodiments, at least one of autonomously comparing the identified eating disorder associated with each patient among the plurality of patients with the received diagnosis of each patient performed by the one or more clinicians or autonomously modifying the one or more of the first set of weighted values, the second set of weighted values, the third set of weighted values, the fourth set of weighted values, the fifth set of weighted values, the first constant value associated with diagnosis of the first eating disorder, the second constant value associated with diagnosis of the second eating disorder, the third constant value associated with diagnosis of the third eating disorder, the fourth constant value associated with diagnosis of the fourth eating disorder, or the fifth constant value associated with diagnosis of the fifth eating disorder might be performed in near-real-time.

In some embodiments, the set of questions might comprise a first category of questions, a second category of questions, and a third category of questions. In some cases, the first category of questions might comprise questions regarding conditions including at least one of body-mass index (“BMI”), weight loss during the previous year, or self-induced vomiting, and/or the like. In some instances, the second category of questions might comprise questions regarding behavior including at least one of eating patterns, dieting, weighing one's self, isolation from friends and family, or avoiding activities, and/or the like. In some cases, the third category of questions might comprise questions regarding thoughts including at least one of being afraid of losing control over eating, thoughts about food, believing one's self to be fat when others call one too thin, or reaction to weight gain, and/or the like. In some instances, the first set of weighted values might be differently defined based on differences among the first category of questions, the second category of questions, and the third category of questions.

Various modifications and additions can be made to the embodiments discussed without departing from the scope of the invention. For example, while the embodiments described above refer to particular features, the scope of this invention also includes embodiments having different combination of features and embodiments that do not include all of the above described features.

### Specific Exemplary Embodiments

We now turn to the embodiments as illustrated by the drawings. FIGS. 1-6 illustrate some of the features of the method, system, and apparatus for implementing eating disorder diagnosis, and, more particularly, to methods, systems, and apparatuses for implementing eating disorder diagnosis based on analysis of patient responses to a set of questions having closed-ended answer options, as referred to above. The methods, systems, and apparatuses illustrated by FIGS. 1-6 refer to examples of different embodiments that include various components and steps, which can be considered alternatives or which can be used in conjunction with one another in the various embodiments. The description of the illustrated methods, systems, and apparatuses shown in FIGS. 1-6 is provided for purposes of illustration and should not be considered to limit the scope of the different embodiments.

FIG. 1 is a schematic diagram illustrating a system 100 for implementing eating disorder diagnosis, in accordance with various embodiments.

In the non-limiting embodiment of FIG. 1, system 100 might comprise a computing system 105a and a data store or database 110a that is local to the computing system 105a. In some cases, the database 110a might be external, yet communicatively coupled, to the computing system 105a. In other cases, the database 110a might be integrated within the computing system 105a. System 100, according to some embodiments, might further comprise one or more user devices 115 that may be used by a patient 120, the one or more user devices 115 comprising a display screen(s) 115a. In some cases, the one or more user devices 115 might include, without limitation, at least one of a tablet computer, a smart phone, a mobile phone, a laptop computer, a desktop computer, or a monitor, and/or the like.

In some embodiments, system 100 might further comprise one or more user devices 125 associated with or used by one or more healthcare professionals or medical practitioners 130. In some instances, the one or more healthcare professionals or medical practitioners 130 might each include, but is not limited to, one of a general medical practitioner, a primary care physician, a psychiatrist, a clinician, or a nurse, and/or the like. In some cases, the user device 125 associated with the one or more healthcare professionals or medical practitioners 130 might include, without limitation, one of a tablet computer, a smart phone, a mobile phone, a laptop computer, or a desktop computer, and/or the like. The user device(s) 125 might communicatively couple with computing system 105a via network(s) 135, while the user device(s) 115 might communicatively couple with computing system 105a via wired communications (depicted by connecting line between the computing system 105a and the user device(s) 115) or wireless communications (depicted by the lightning bolt symbol between the computing system 105a and the user device(s) 115).

According to some embodiments, system 100 might further comprise a medical server(s) 140 and corresponding database(s) 145 that communicatively couples with user device(s) 125 and/or computing system 105a via network(s) 135. In some cases, alternative or additional to the computing system 105a and corresponding database 110a being located proximate to the user device(s) 115 and/or the patient 120, system 100 might comprise remote computing system 105b and corresponding database(s) 110b that communicatively couple with the one or more user devices 115 via the one or more networks 135. In some embodiments, remote computing system 105b might comprise at least one of a server computer over a network, a cloud-based computing system over a network, and/or the like.

In operation, computing system 105a, computing system 105b, user device(s) 115, user device(s) 125, and/or medical server(s) 140 (collectively, “computing system”) might receive, from a first patient 120 (e.g., via the user device(s) 115 or via a user interface device (such as a voice input, a touch input, a key or button input, or the like) of the user device(s) 115, or the like), a first set of patient responses 155 to a set of questions 150 each having closed-ended answer options. Each question 150 might be dichotomized such that a first set of answer options among its closed-ended answer options are assigned a first score while a second set of answer options among its closed-ended answer options are assigned a second score.

In some embodiments, the set of questions 150 might include, without limitation, a first category of questions, a second category of questions, and a third category of questions, or the like. The first category of questions might comprise questions regarding conditions including, but not limited to, at least one of body-mass index (“BMI”), weight loss during the previous year, or self-induced vomiting, and/or the like. The second category of questions might comprise questions regarding behavior including, without limitation, at least one of eating patterns, dieting, weighing one's self, isolation from friends and family, or avoiding activities, and/or the like. The third category of questions might comprise questions regarding thoughts including, but not limited to, at least one of being afraid of losing control over eating, thoughts about food, believing one's self to be fat when others call one too thin, or reaction to weight gain, and/or the like. The first set of weighted values might be differently defined based on differences among the first category of questions, the second category of questions, and the third category of questions. The computing system might determine, for each dichotomized question among the set of dichotomized questions, whether a corresponding patient response among the first set of patient responses corresponds to the first set of answer options for that dichotomized question or corresponds to the second set of answer options for that dichotomized question, wherein the first set of answer options for the set of dichotomized questions is indicative of likelihood of patients selecting such answer options having an eating disorder, and wherein the second set of answer options for the set of dichotomized questions is indicative of likelihood of patients selecting such answer options not having an eating disorder. The computing system might determine a diagnosis of whether or not the first patient has an eating disorder, based at least in part on logistic regression analysis of one or more of the patient responses among the first set of patient responses corresponding to the first set of answer options for the set of dichotomized questions or the patient responses among the first set of patient responses corresponding to the second set of answer options for the set of dichotomized questions.

Based on a determination that the first patient likely has an eating disorder, the computing system might perform one or more of the following: (a) identify a first set of weighted values each corresponding to each of the received first set of patient responses from the first patient, and calculate a first probability of diagnosis of a first eating disorder, based at least in part on modification of the received first set of patient responses by multiplication with the first set of weighted values and by subsequent addition of a first constant value associated with diagnosis of the first eating disorder; (b) identify a second set of weighted values each corresponding to each of the received first set of patient responses from the first patient, and calculate a second probability of diagnosis of a second eating disorder, based at least in part on modification of the received second set of patient responses by multiplication with the second set of weighted values and by subsequent addition of a second constant value associated with diagnosis of the second eating disorder; (c) identify a third set of weighted values each corresponding to each of the received first set of patient responses from the first patient, and calculate a third probability of diagnosis of a third eating disorder, based at least in part on modification of the received third set of patient responses by multiplication with the third set of weighted values and by subsequent addition of a third constant value associated with diagnosis of the third eating disorder; (d) identify a fourth set of weighted values each corresponding to each of the received first set of patient responses from the first patient, and calculate a fourth probability of diagnosis of a fourth eating disorder, based at least in part on modification of the received fourth set of patient responses by multiplication with the fourth set of weighted values and by subsequent addition of a fourth constant value associated with diagnosis of the fourth eating disorder; and/or (e) identify a fifth set of weighted values each corresponding to each of the received first set of patient responses from the first patient, and might calculate a fifth probability of diagnosis of a fifth eating disorder, based at least in part on modification of the received fifth set of patient responses by multiplication with the fifth set of weighted values and by subsequent addition of a fifth constant value associated with diagnosis of the fifth eating disorder; and/or the like. In some embodiments, the first eating disorder, the second eating disorder, the third eating disorder, the fourth eating disorder, and the fifth eating disorder might each comprise one of anorexia nervosa (“AN”), bulimia nervosa (“BN”), binge-eating disorder (“BED”), obesity (“OB”), or other specified feeding or eating disorder (“OSFED”), and/or the like.

The computing system might identify which eating disorder the first patient is likely to have, based at least in part on the determined diagnosis and based at least in part on the calculated first through fifth probabilities. In some embodiments, the computing system might identify suggested therapy techniques for the identified eating disorder. In some cases, the computing system might display, on a display device (e.g., on display screen(s) 115a of user device(s) 115, or the like), the identified eating disorder and/or the identified suggested therapy techniques for the identified eating disorder. In some cases, displaying the identified eating disorder might comprise displaying, with the computing system, the identified eating disorder in a software application (“app”) running on the display device. In some embodiments, the computing system might send a message to user device(s) 125 associated with the clinician or healthcare professional 130, the message comprising the identified eating disorder associated with the first patient 120. In some instances, the message might further comprise the identified suggested therapy techniques associated with the identified eating disorder associated with the first patient.

According to some embodiments, such as during the development phase, during periodic or occasional training periods, or for periodic or occasional enhancement of the autonomous diagnosis functionalities, and/or the like, the computing system might receive diagnosis of the first patient 120 performed by a clinician or healthcare professional 130 among the one or more healthcare professionals 130. The computing system might compare the identified eating disorder of the first patient with the received diagnosis of the first patient performed by the clinician or healthcare professional 130 to determine whether the identified eating disorder matches the received diagnosis. In some cases, the computing system might further display, on the display device (e.g., on display screen(s) 115a of user device(s) 115, or the like), the received diagnosis of the first patient 120 performed by the clinician or healthcare professional 130. In some embodiments, the computing system might receive a plurality of patient responses associated with a plurality of patients, an identified eating disorder associated with each patient among the plurality of patients, and a diagnosis of each patient among the plurality of patients performed by one or more clinicians or healthcare professionals 130. The computing system might compare the identified eating disorder associated with each patient among the plurality of patients with the received diagnosis of each patient performed by the one or more clinicians or healthcare professionals 130 to determine whether the identified eating disorder matches the received diagnosis. Based on the comparison, the computing system might analyze the plurality of sets of patient responses associated with the plurality of patients, the identified eating disorder associated with each patient among the plurality of patients, the diagnosis of each patient among the plurality of patients performed by the one or more clinicians or healthcare professionals 130, and one or more of the first through fifth set of weighted values to determine whether one or more of the first set of weighted values, the second set of weighted values, the third set of weighted values, the fourth set of weighted values, the fifth set of weighted values, the first constant value associated with diagnosis of the first eating disorder, the second constant value associated with diagnosis of the second eating disorder, the third constant value associated with diagnosis of the third eating disorder, the fourth constant value associated with diagnosis of the fourth eating disorder, or the fifth constant value associated with diagnosis of the fifth eating disorder are optimal or should be updated or modified, and/or the like.

The computing system might modify one or more of the first set of weighted values, the second set of weighted values, the third set of weighted values, the fourth set of weighted values, the fifth set of weighted values, the first constant value associated with diagnosis of the first eating disorder, the second constant value associated with diagnosis of the second eating disorder, the third constant value associated with diagnosis of the third eating disorder, the fourth constant value associated with diagnosis of the fourth eating disorder, or the fifth constant value associated with diagnosis of the fifth eating disorder, based at least in part on one or more of the comparison of the identified eating disorder associated with each patient among the plurality of patients (including the first patient 120) with the received diagnosis of each patient performed by the one or more clinicians or healthcare professionals 130 and/or the received plurality of patient responses associated with the plurality of patients. In other words, as more and more patients are correctly or incorrectly diagnosed or classified in terms of eating disorders that the patients might have, the computing system may be further trained to more accurately diagnose or classify future eating disorders in future patients. According to some embodiments, to avoid training biases, the weights or coefficients and constants may be reset every N number of patients, where N is a predetermined number of patients.

In some aspects, the reliability and validity of psychiatric diagnoses for those individuals with eating disorders have been quantified. In 2012, 37 out of 174 (or 21%) patients were referred by a psychiatrist to the Mandometer Clinic and 20 out of 37 (or 54%) of these patients had been diagnosed with a mental disorder (i.e., depression, anxiety, ADHD, etc.) in addition to an eating disorder. To examine the reliability and validity of these diagnoses, the structured Clinical Interview for DSM-IV, Axis I Disorders (“SCID-I”) was administered to all 174 patients by clinical staff trained for that purpose. Of the 174 patients, 120 (or 69%) patients scored high enough to indicate the existence of a psychiatric diagnosis, with anxiety (44%) and depression (32%) being the most common diagnoses emerging from the SCID-I interview. There was agreement between the diagnosis made by a psychiatrist and the SCID-I results in only 17 out of 37 (or 46%) cases, a proportion that was not statistically significant. When the 174 patients were given the opportunity to see a psychiatrist, 60 patients (or 34%) opted for such care. There was agreement between the outcome of the SCID-I interview and the patient's subjective experience of a psychiatric problem was found in only 64 out of 174 (or 37%) of the cases, indicating significant disagreement between the outcome of the SCID-I interview and the patient's subjective experience of a psychiatric problem.

The reliability and validity of the diagnoses of psychiatric syndromes among patients with eating disorders are therefore questionable. Reducing the problems of misdiagnosis can direct patients to the proper psychiatric treatments.

About 20% of all eating disorders patients assessed for treatment at the Mandometer Clinics in Sweden are referred by general practitioners, primary care physicians, or psychiatrists, with 80% being online self-referrals by patient or parents. The agreement between the diagnosis by the physician and the diagnosis at a Mandometer clinic is about 80% for anorexia nervosa (“AN”), about 37% for bulimia nervosa (“BN”), and about 26% for binge-eating disorder (“BED”). Of the other diagnoses, 20% of the anorexic patients had been diagnosed as having an eating disorder not otherwise specified (“EDNOS”), 63% of the bulimic patients had been diagnosed with AN or EDNOS, and 13% of the BED patients had been diagnosed as BN, 19% as EDNOS and 42% as obesity (“OB”).

Of 93 patients, 88 participated in a 2018 survey concerning the diagnosis they considered to be their main problem. Half of these patients considered their main problem to be AN, a fifth considered their problem to be BN, and about a third thought the problem was EDNOS. When evaluated clinically, the patients had interpreted their EDNOS problems as AN-problems. In addition, 11% of the patients thought that they had both AN and BN, and 20% thought that they had neither AN nor BN. These patients are included among the EDNOS patients.

The various embodiments provide for techniques and systems for implementing eating disorder diagnosis based on analysis of patient responses to a set of questions having closed-ended answer options. Such eating disorder diagnosis, as described in greater detail below with respect to the figures, result in optimized, relatively quick, accurate, and objective diagnosis or classification of patient eating disorders (as opposed to imprecise and/or subjective diagnosis or classification by differing clinicians or healthcare providers, who may or may not be influenced by their training or limited (or different) experiences), which would allow for more targeted or tailored treatment of such eating disorders for each particular patient, and/or the like. Further, because a nurse, social worker, or receptionist can hand out user devices (e.g., tablet computers, etc.) that a subject or patient can fill out and that can either perform the processes of the computing system as described with respect to FIG. 1 or the like, or that can send the patient responses to such computing system, the various embodiments can free up doctors to oversee the diagnosis of patients whose identified or diagnosed eating disorders are ambiguous or inconclusive (e.g., as in the case with patients having multiple eating disorders, or the like). As more and more patients are correctly or incorrectly diagnosed or classified in terms of eating disorders that the patients might have, the computing system may be further trained to more accurately diagnose or classify future eating disorders in future patients. In some embodiments, to avoid training biases, the weights or coefficients and constants may be reset every N number of patients, where N is a predetermined number of patients.

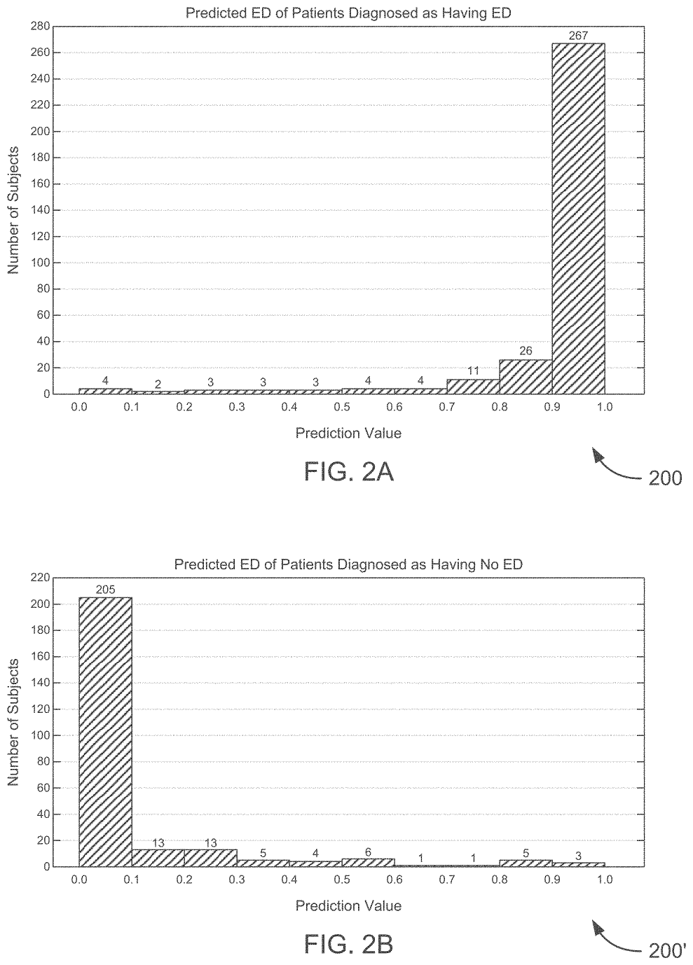

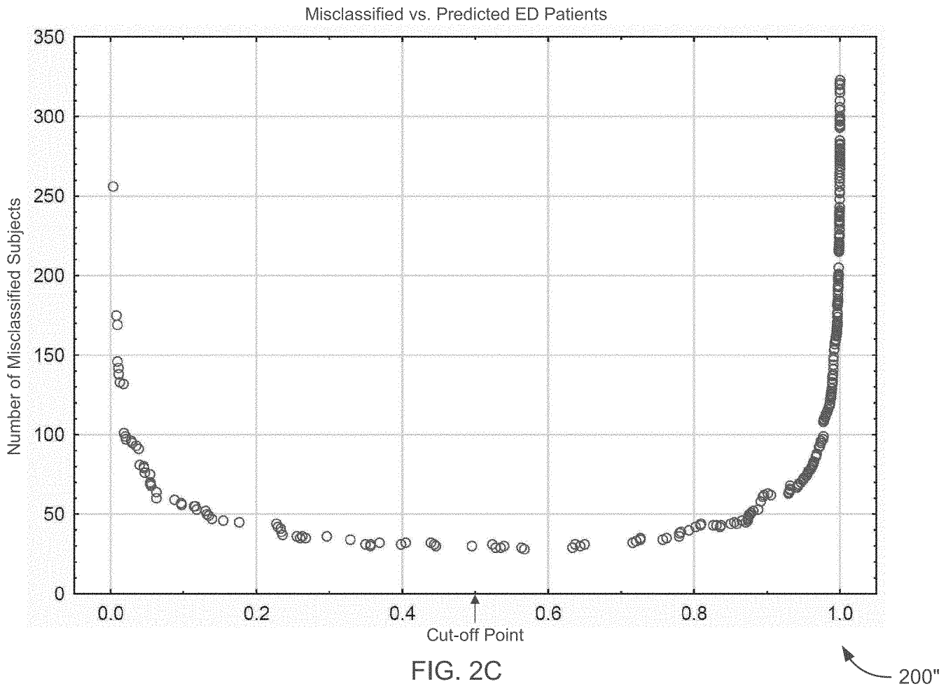

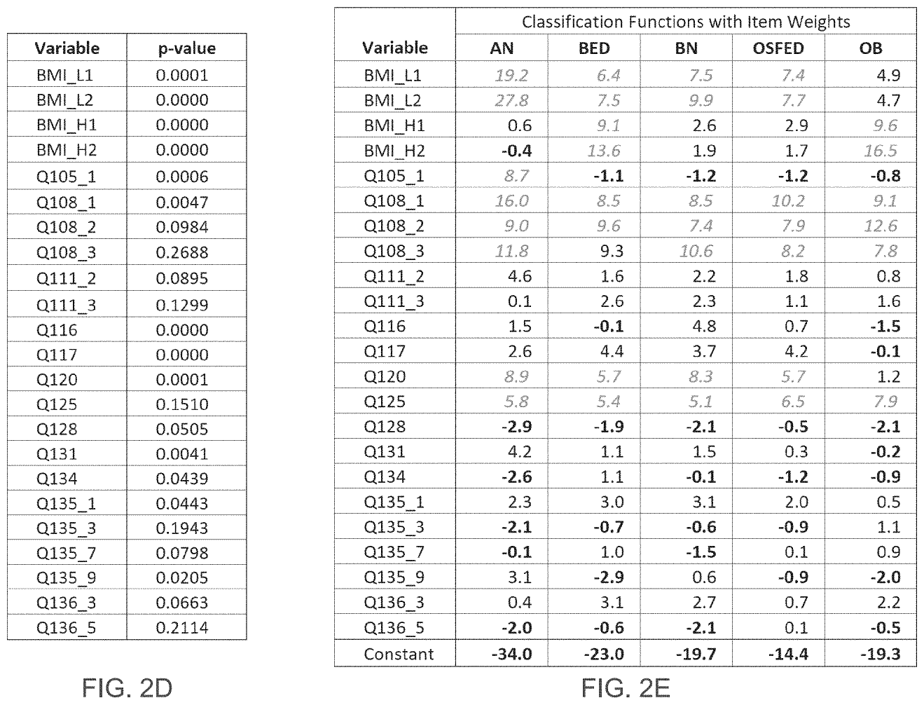

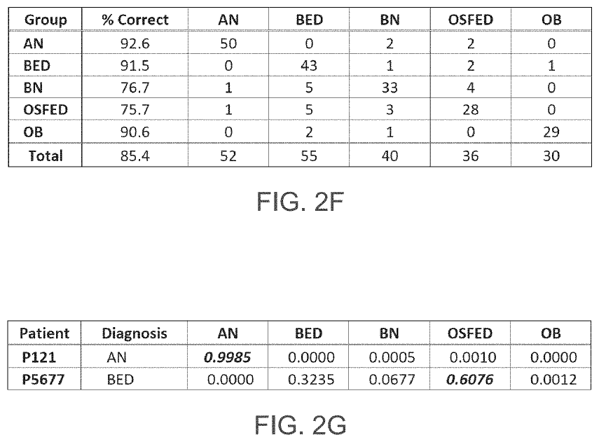

These and other functions of the system 100 (and its components) are described in greater detail below with respect to FIGS. 2-4.

FIGS. 2A-2G (collectively, “FIG. 2”) illustrate non-limiting examples of graphs and tables depicting predicted eating disorders, misclassifications of eating disorders, weightings and constants for eating disorders, and percentage of correct diagnoses of eating disorders, or the like. FIGS. 2A and 2B are graphical diagrams illustrating non-limiting examples 200 and 200′ of estimated probabilities of eating disorders for subjects diagnosed as having eating disorders and for subjects diagnosed as having no eating disorders, respectively, in response to the implemented eating disorder diagnosis, in accordance with various embodiments. FIG. 2C is a graphical diagram illustrating a non-limiting example 200″ of misclassification of eating disorders versus predicted eating disorders, in accordance with various embodiments. FIGS. 2D and 2E illustrate non-limiting examples of tables listing p-values for corresponding variables and listing classification functions with item weights for each eating disorder, respectively, in accordance with various embodiments. FIGS. 2F and 2G illustrate non-limiting examples of tables listing the percentage of correct diagnoses of eating disorders for a group of individuals and listing percentage of correct diagnosis of eating disorder for particular patients, in accordance with various embodiments.

With reference to FIG. 2A, a bar graph is shown depicting the number of subjects or patients for each set of prediction values 0.0-1.0 in 0.1 increments, where the prediction values might correspond to probability values that estimate likelihood of the patients being diagnosed as having an eating disorder, with 0.0 corresponding to 0% and 1.0 corresponding to 100%. In particular, as shown in the non-limiting example 200 of FIG. 2A, in accordance with the various embodiments, for the following prediction values, the system (such as the computing system of FIG. 1, or the like) might predict that the corresponding number of patients (in parentheses) might be diagnosed as having an eating disorder: 0.0-0.1 (4 subjects or patients); 0.1-0.2 (2 subjects or patients); 0.2-0.3 (3 subjects or patients); 0.3-0.4 (3 subjects or patients); 0.4-0.5 (3 subjects or patients); 0.5-0.6 (4 subjects or patients); 0.6-0.7 (4 subjects or patients); 0.7-0.8 (11 subjects or patients); 0.8-0.9 (26 subjects or patients); and 0.9-1.0 (267 subjects or patients).

Similarly, referring to FIG. 2B, a bar graph is shown depicting the number of subjects or patients for each set of prediction values 0.0-1.0 in 0.1 increments, where the prediction values might correspond to probability values that estimate likelihood of the patients being diagnosed as not having an eating disorder, with 0.0 corresponding to 0% and 1.0 corresponding to 100%. In particular, in the embodiment 200′ of FIG. 2B, in accordance with the various embodiments, for the following prediction values, the system (such as the computing system of FIG. 1, or the like) might predict that the corresponding number of patients (in parentheses) might be diagnosed as not having an eating disorder: 0.0-0.1 (205 subjects or patients); 0.1-0.2 (13 subjects or patients); 0.2-0.3 (13 subjects or patients); 0.3-0.4 (5 subjects or patients); 0.4-0.5 (4 subjects or patients); 0.5-0.6 (6 subjects or patients); 0.6-0.7 (1 subjects or patients); 0.7-0.8 (1 subjects or patients); 0.8-0.9 (5 subjects or patients); and 0.9-1.0 (3 subjects or patients).

Turning to FIG. 2C, a graph is shown depicting the number of misclassified subjects or patients versus the predicted eating disorder patients, in accordance with the various embodiments. In some embodiments, a chosen optimal cutoff limit of 0.5 might minimize the number of falsely classified subjects or patients, for example, 15 subjects or patients estimated as not having eating disorders based on this cutoff limit in FIG. 2A and 16 subjects or patients estimated as having eating disorders based on this cutoff limit in FIG. 2B, out of a total number of 583 subjects or patients. Based on this cutoff, the misclassification rate is 31 out of 583 or less than 6%.

In view of FIGS. 2A-2C, for prediction of eating disorders, with p corresponding to the prediction value or probability: p≥0.9 might indicate distinct or marked eating disorder; 0.7≤p<0.9 might indicate eating disorder; 0.5<p<0.7 might indicate possible eating disorder; 0.3<p<0.5 might indicate possible no eating disorder; 0.1<p≤0.3 might indicate no eating disorder; and p≤0.1 might indicate distinct or marked no eating disorder.

Merely by way of example, in some cases, the prediction of whether the subjects or patients each has an eating disorder may be based on analysis of a set of patient responses (by each patient) to a set of questions each having closed-ended answer options. For instance, the set of questions might include, without limitation, questions regarding: (1) gender of a patient; (2) age or date of birth of the patient; (3) height of the patient; (4) weight of the patient; (5) whether the patient's body weight has changed (i.e., gained, lost, or not changed) during the previous year (and if so, by how much, e.g., less than 11 pounds (or 5 kg), between 11 and 22 pounds (or 5-10 kg), or more than 22 pounds (or 10 kg)); (6) how much the patient would like to weigh; (7) whether the patient has ever been afraid of not being able to stop eating once the patient has started eating (e.g., yes, no, or don't know); (8) the patient's pattern of eating (e.g., eating regular meals, limiting food intake to lose weight or avoid weight gain, starving the patient periodically and limiting food intake between those periods, overeating periodically and limiting food intake between those periods, overeating daily or almost daily, binge-eating periodically and limiting food intake between those periods, binge-eating daily or almost daily, or the like); (9) whether the patient has ever induced vomiting after having eaten (e.g., no, never; yes, occasionally; yes, often; yes, regularly; or yes, always; etc.); (10) estimating the patient's fear of gaining weight (e.g., extremely strong, strong, moderate, mild, or none, etc.); (11) which meal the patient usually eats (e.g., breakfast, lunch, and/or dinner, and for each always, sometimes, or never, etc.); (12) how often the patient snacks in between meals (e.g., never between meals; periodically and eating regular meals between those periods; sometimes between meals; always between meals; or all day and never eating any regular meals; etc.); (13) how fast or slow the patient eats a meal compared to others (e.g., much faster than others; somewhat faster than others; much slower than others; somewhat slower than others; or neither faster nor slower than others; etc.); and (14) how much the patient eats compared to others (e.g., much more than others; somewhat more than others; much less than others; somewhat less than others; or neither more nor less than others; etc.).

In some embodiments, the set of questions might further include, without limitation, questions regarding: (15) how often the patient weighs themselves (e.g., several times daily; daily; 2-3 times a week; monthly; or yearly or less; etc.); (16) whether the patient ever makes themselves vomit because he, she, or they feel uncomfortably full (e.g., yes or no); (17) whether the patient worries over having lost control over how much the patient eats (e.g., yes or no); (18) whether the patient has lost more than one stone (i.e., 14 pounds or 6 kg) in a three-month period (e.g., yes or no); (19) whether the patient believes themselves to be fat when others say the patient is too thin (e.g., yes or no); (20) whether food dominates the patient's life (e.g., yes or no); (21) whether the patient is satisfied with his, her, or their current weight (optional); (22) whether the patient has ever exercised to burn calories (optional); (23) whether the patient has a weight problem (e.g., yes or no); (24) whether the patient is satisfied with his, her, or their body appearance (e.g., yes or no); (25) whether the patient has ever lost weight (e.g., yes or no); (26) how much weight the patient lost during the previous three months (e.g., less than 11 pounds (or 5 kg); between 11 and 22 pounds (or 5-10 kg); more than 22 pounds (or 10 kg); etc.); (27) whether, in the patient's opinion, the patient has ever been at a completely normal weight (e.g., now; earlier in the patient's life; both now and earlier in the patient's life; or never; etc.); and (28) whether the patient has ever regurgitated food by re-chewing, re-swallowing, or spitting out the food (e.g., yes or no).

According to some embodiments, the set of questions might further include, without limitation, questions regarding: (29) whether the patient's eating behavior has caused the patient to isolate him, her, or themselves from friends and family (e.g., yes or no); (30) whether the patient's eating behavior caused the patient to avoid or interrupt activities that the patient would otherwise like to perform (e.g., yes or no); (31) how the patient would react if the patient gained 4.4 pounds (or 2 kg) (e.g., will panic; will start to eat less food, but will not panic; or would not bother the patient; etc.); (32) whether the patient ever eats alone because the patient is ashamed of how much food the patient eats (e.g., yes or no); (33) whether the patient has regular meal habits (e.g., yes, at least three regular meals per day; yes, at least one regular meal per day; no regular meals; etc.); (34) whether the patient ever avoids looking at himself, herself, or themselves in the mirror (e.g., never; yes, occasionally; or yes, always; etc.); (35) how much time (in hours per day, or 0=no time, 1=only at meal times, and 2=all waking time) that the patient spends on average on eating-related behaviors (e.g., thinking about food; reading recipes or cook books; reading nutrition labels; shopping for food; planning meals; cooking; eating; baking; being in a kitchen to watch others cook; hoarding food; etc.); and whether the patient has any of the particular eating or weight issues (e.g., excessive dieting; self-starvation; binge-eating; overweight; or obesity; etc.); etc.

To improve the reliability and validity of diagnosis of eating disorders, the various embodiments have been developed to assess the set of questions, each with a closed-ended answering options that are presented on a user device (e.g., user device(s) 115 of FIG. 1, or the like). In a first trial, 257 healthy volunteers and 338 eating disorder patients, who were referred by a physician or were self-referred for treatment, had answered a preliminary questionnaire with 20 questions. In a second trial, 252 referred eating disorder patients had answered an extended questionnaire with 34 questions with closed-ended answering options (e.g., the 34 of the 36 questions (1)-(36), excluding the optional questions (21) and (22) above, or the like).

A logistic regression analysis of the patients' responses to the set of questions yielded a probability for an eating disorder versus no eating disorder. A first cutoff limit (“C1”) was chosen to minimize the number of falsely classified individuals. Patients scoring below C1 were thus classified as false-negative (who should be treated) and healthy individuals scoring above C1 were classified as false-positive (who should not be treated). Scores below a second cutoff limit (“C2”), on the other hand, might identify individuals who do not have an eating disorder. Patients scoring between C1 and C2 might be classified as having a mild eating disorder and those patients scoring above C1 might be classified as likely having an eating disorder. These cutoff limits might minimize the risk of missing someone who should be diagnosed with an eating disorder (i.e., generates as few false positives as possible). Scores between C1 and C2 might require a second opinion by a clinician before the patient is referred to general psychiatric care or specialist medical care.

In some embodiments, responses for each of the set of questions might be dichotomized to generate a score of 0 or 1 (or some other dichotomized values (e.g., 1 or 2; Y or N; ED or No ED; etc.), with each question or response to that question being assigned a weighted value according to the logistic regression. The sum of the weighted scores may be converted to a probability of having an eating disorder. For instance, with respect to question (10), the first three answer options (i.e., extremely strong, strong, and moderate) might be dichotomized to a first value (e.g., 0, or the like), while the last two answer options (i.e., mild and none) might be dichotomized to a second value (e.g., 1, or the like). The answer options of the other questions may similarly be dichotomized.

According to some embodiments, the set of questions might comprise a first category of questions, a second category of questions, and a third category of questions. In some cases, the first category of questions might comprise questions (e.g., questions (3)-(5), (9), (16), (18), and (26) above, or the like) regarding conditions including at least one of body-mass index (“BMI”), weight loss during the previous year, or self-induced vomiting, and/or the like. In some instances, the second category of questions might comprise questions (e.g., questions (8), (11)-(15), (25), (28)-(30), and (32)-(35) above, or the like) regarding behavior including at least one of eating patterns, dieting, weighing one's self, isolation from friends and family, or avoiding activities, and/or the like. In some cases, the third category of questions might comprise questions (e.g., questions (6), (7), (10), (17), (19), (20), (23), (24), (27), (31), and (36) above, or the like) regarding thoughts including at least one of being afraid of losing control over eating, thoughts about food, believing one's self to be fat when others call one too thin, or reaction to weight gain. In some instances, the first through fifth sets of weighted values (as described above with respect to FIG. 1, or the like) might be differently defined based on differences among the first category of questions, the second category of questions, and the third category of questions, or the like. The three categories of items may be analyzed using three discriminant analyses or so, and then combined into a final result.

FIG. 2D depicts a table illustrating a discriminant function analysis summary in which for particular questions that have been dichotomized (e.g., questions (5), (8), (11), (16), (17), (20), (25), (28), (31), (34), (35), or (36) above, or the like, that have been dichotomized) p-values are listed, based on assessment of 213 subjects or patients. Referring to FIG. 2D, BMI between 17.5 and 19 kg/m² (“BMI_L1”) might have a p-value of 0.0001, while each of BMI less than or equal to 17.5 (“BMI_L2”), BMI between 24 and 30 (“BMI H1”), and BMI greater than or equal to 30 (“BMI H2”) might have a p-value of 0.0000.

The following responses to particular questions above (denoted in FIG. 2D as variables) might have the following p-values, based on assessment of the 213 subjects or patients: Q105_1 (response to question (5) indicating that the patient has gained weight) with a p-value of 0.0006; Q108_1 (response to question (8) indicating that the patient eats regular meals) with a p-value of 0.0047; Q108_2 (response to question (8) indicating that the patient limits food intake to lose weight or to avoid weight gain) with a p-value of 0.0984; Q108_3 (response to question (8) indicating that the patient starves himself, herself, or themselves periodically and limits food intake between those periods) with a p-value of 0.2688; Q111_2 (response to question (11) indicating that the patient usually eats lunch always or sometimes) with a p-value of 0.0895; Q111_3 (response to question (11) indicating that the patient usually eats dinner always or sometimes) with a p-value of 0.1299; Q116 (response to question (16) indicating that the patient makes himself, herself, or themselves vomit because the patient felt uncomfortably full) with a p-value of 0.0000; Q117 (response to question (17) indicating that the patient worries that he, she, or they have lost control over how much the patient eats) with a p-value of 0.0000; Q120 (response to question (20) indicating that the patient believes that food dominates the patient's life) with a p-value of 0.0001; Q125 (response to question (25) indicating that the patient has ever lost weight) with a p-value of 0.1510; Q128 (response to question (28) indicating that the patient has ever regurgitated food) with a p-value of 0.0505; Q131 (response to question (31) indicating that the patient will panic if he, she, or they gained 4.4 pounds (or 2 kg)) with a p-value of 0.0041; Q134 (response to question (34) indicating that the patient has ever avoided looking at himself, herself, or themselves in the mirror) with a p-value of 0.0439; Q135_1 (response to question (35) indicating that the patient spends time thinking about food) with a p-value of 0.0443; Q135_3 (response to question (35) indicating that the patient spends time reading nutrition labels) with a p-value of 0.1943; Q135_7 (response to question (35) indicating that the patient spends time eating) with a p-value of 0.0798; Q135_1 (response to question (35) indicating that the patient spends time being in a kitchen to watch others cook) with a p-value of 0.0205; Q136_3 (response to question (36) indicating that the patient binge-eats) with a p-value of 0.0663; and Q136_5 (response to question (36) indicating that the patient has obesity issues) with a p-value of 0.2114; or the like.

With reference to FIG. 2E, a table is shown listing classification functions with item weights or discriminant function coefficients. In particular, with respect to the variables listed in FIGS. 2D and 2E, weighted values are listed for each variable for each classification of eating disorder (e.g., AN, BED, BN, OSFED, or OB, etc.). As depicted in FIG. 2E, weighted values greater than 5 might be italicized (e.g., weighted value of 19.2 for BMI_L1 for AN, or the like), while negative values (indicative of a reverse or inverse influence on the discrimination) might be bold-faced (e.g., weighted value of −1.2 for Q105_1 for BN, or the like). In addition to the weighted values, each classification of eating disorder might have a constant value associated with diagnosis of the particular eating disorder (e.g., constant values of −34.0, −23.0, −19.7, −14.4, and −19.3 for AN, BED, BN, OSFED, and OB, respectively, or the like).

According to some embodiments, the discriminant function coefficients as shown in the table of FIG. 2E are multiplied by the patient's dichotomized answer vector (as described above with respect to FIG. 1). The result of this calculation is then added to the constant values (shown in the table of FIG. 2E) to form a score for each of the five diagnoses (which may be denoted, Sc(i), where i=1, . . . , 5 for each of the five eating disorders, AN, BN, BED, OB, and OSFED), and then is assigned the diagnosis corresponding to the highest score. The accuracy of the assignment, diagnosis, or classification is then estimated by an approximate probability:

Prob(diagnose i)=e^{Sc(i)}/Σᵢ₌₁⁵e^{Sc(i)} (Eq. 1)

The calculated error is <0.01. In case there are two or more calculated probabilities that are close to each other (e.g., less than 0.3 difference), the fact that the assignment, diagnosis, or classification is not conclusive will be reported to the clinician.

Referring to FIG. 2F, a classification matrix is shown that compares clinical diagnosis (shown in the rows of the table) with diagnosis in accordance with the various embodiments (shown in the columns of the table). The diagnoses are performed according to diagnostic and statistical manual of mental disorders (“DSM-5”). As shown in FIG. 2F, in a set of trial diagnoses, for a first plurality of patients who are clinically diagnosed with AN, the system (e.g., the computing system as described above with respect to FIG. 1, or the like) might correctly diagnose or classify 50 patients with AN, while incorrectly diagnosing 2 patients with BN and 2 patients with OSFED, resulting in an accuracy score of 92.6% correct. In the set of trial diagnoses, for a second plurality of patients who are clinically diagnosed with BED, the system might correctly diagnose or classify 43 patients with BED, while incorrectly diagnosing 1 patient with BN, 2 patients with OSFED, and 1 patient with OB, resulting in an accuracy score of 91.5% correct. In the set of trial diagnoses, for a third plurality of patients who are clinically diagnosed with BN, the system might correctly diagnose or classify 33 patients with BN, while incorrectly diagnosing 1 patient with AN, 5 patients with BED, and 4 patients with OSFED, resulting in an accuracy score of 76.7% correct. In the set of trial diagnoses, for a fourth plurality of patients who are clinically diagnosed with OSFED, the system might correctly diagnose or classify 28 patients with OSFED, while incorrectly diagnosing 1 patient with AN, 5 patients with BED, and 3 patients with BN, resulting in an accuracy score of 75.7% correct. In the set of trial diagnoses, for a fifth plurality of patients who are clinically diagnosed with OB, the system might correctly diagnose or classify 29 patients with OB, while incorrectly diagnosing 2 patients with BED and 1 patient with BN, resulting in an accuracy score of 90.6% correct. Thus, at least for the set of trial diagnoses, the system has an overall or average accuracy of 85.4% correct.

In another set of trial diagnoses, as shown in FIG. 2G, a table is shown depicting a comparison between clinical diagnosis and diagnosis in accordance with the various embodiments for two particular patients, whose ID numbers are P121 and P5677. As with the set of trial diagnoses underlying the table of FIG. 2F, the diagnoses for P121 and P5677 are performed according to DSM-5. In the another set of trial diagnoses, the system (e.g., the computing system as described above with respect to FIG. 1, or the like) might correctly diagnose or classify patient P121 as having AN (with p-values of 0.9985 for AN, 0.0005 for BN, 0.0010 for OSFED, and 0.0000 for each of BED and OB), which matches the clinical diagnosis of the patient. The system might diagnose or classify patient P5677 as having OSFED (with p-values of 0.6076 for OSFED, 0.3235 for BED, 0.0677 for BN, 0.0012 for OB, and 0.0000 for AN), which differs from the clinical diagnosis of the patient (namely, BED). Here, the system more accurately and more objectively identifies OSFED and BED as main candidate eating disorders that the patient P5677 might have, with BN as a lesser candidate eating disorder that the patient P5677 might have. Due to the complex nature of the patient's eating disorder, the patient might manifest multiple eating disorders, as is the case with patient P5677. Clinicians in general may not be easily able to diagnose or classify multiple eating disorders in patients, and may focus on one rather than all of the multiple eating disorders and may even focus on a lesser eating disorder rather than the primary eating disorder (as in the case with patient P5677).

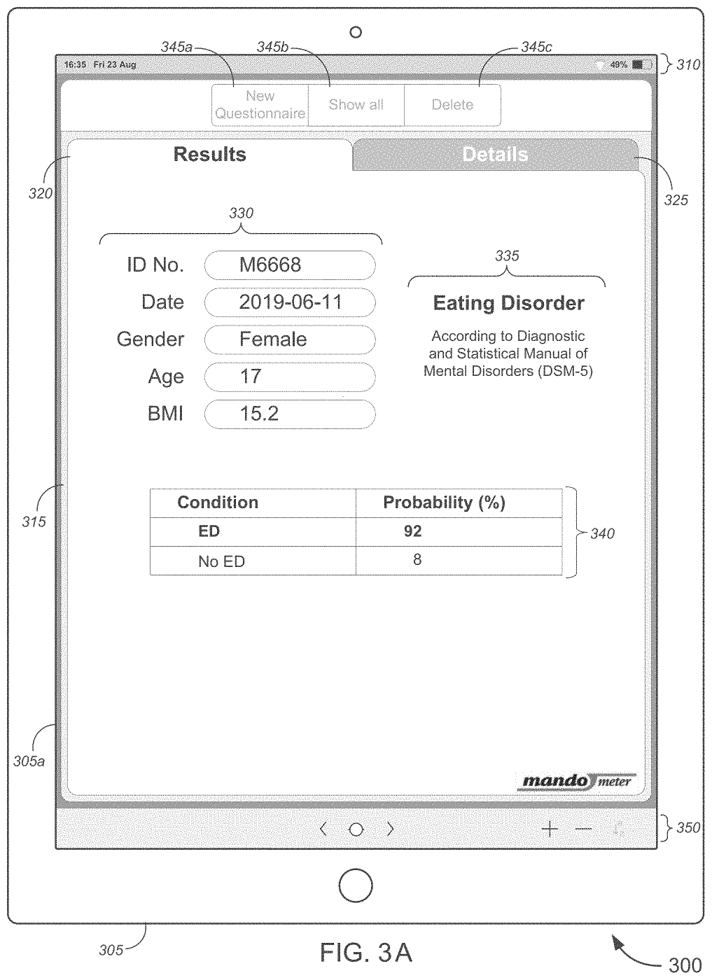

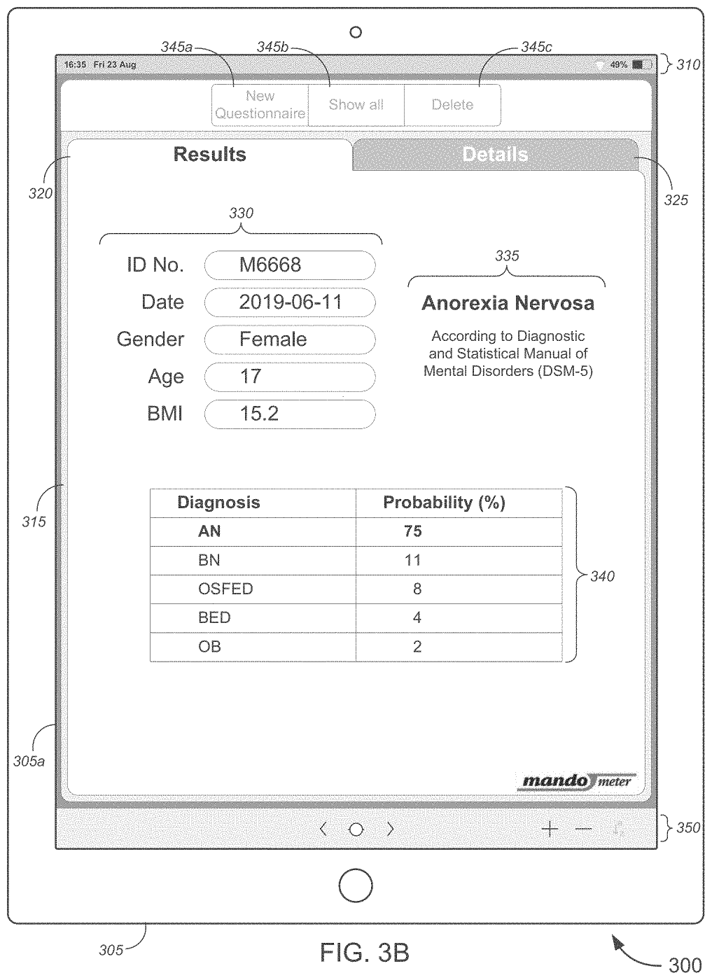

FIGS. 3A and 3B (collectively, “FIG. 3.”) are schematic diagrams illustrating another non-limiting example of a user device 300 that presents an exemplary graphical user interface for displaying diagnosis of eating disorders, in accordance with various embodiments. FIG. 3A depicts display of diagnosis and probability of eating disorder (“ED”) or no eating disorder (“ED”), while FIG. 3B depicts display of diagnoses and probabilities of particular types of eating disorders.

In the non-limiting embodiment of FIG. 3, user device 300 might comprise a housing 305 and a display screen 305a (which might include a touchscreen display screen or a non-touchscreen display screen, or the like). On the display screen 305a might be displayed a user interface or a software application (“app”). The user interface or app might comprise a header portion 310, a main portion 315, a results tab 320 of the main portion 315, and a details tab 325 of the main portion 315. The header portion 310 might include, without limitation, date and time information, network connectivity icon or symbol (e.g., Wi-Fi connectivity icon or symbol, cellular connectivity icon or symbol, Bluetooth™ connectivity icon or symbol, etc.), and battery icon and charge level (in this case, “49%”), or the like. The results tab 320 might include, but is not limited to, patient information portion 330, a diagnosis summary portion 335, and diagnosis summary table portion 340, or the like. In some embodiments, the patient information portion 330 might include, without limitation, a patient identification (“ID”) field (which might display the patient's ID number, in this case, “M6668”), a diagnosis date field (which might display the date of diagnosis, in this case, “2019-06-11”), a gender field (which might display the gender of the patient, in this case, “Female”), an age field (which might display the age of the patient, in this case, “17”), and body mass index (“BMI”) risk field (which might display the patient's BMI risk, in this case, “Yes”; where BMI is calculated by dividing a patient's weight by the patient's height in meters squared (BMI=W/h²); where the BMI risk refers to health risks associated with BMI values below or above the normal BMI range of 18.5-25 kg/m²), or the like. The diagnosis summary portion 335 might display a diagnosis of the patient (in this case, “Eating Disorder” (FIG. 3A) and “Anorexia Nervosa” (FIG. 3B)) based on the analysis performed by the system, in accordance with the various embodiments, and might display information regarding the diagnosis being determined according to Diagnostic and Statistical Manual of Mental Disorders (“DSM-According to some embodiments, with reference to the non-limiting example of FIG. 3A, the diagnosis summary table portion 340 might display a table depicting the probability (in percentage values) of the likelihood the patient is diagnosed with either having at least one eating disorder (“ED”; in this case, 92%”) or having no eating disorder (“No ED”; in this case, 8%). In this case, as the probability of the patient having at least one eating disorder is an overwhelming 92%, ED and 92% are highlighted in boldface, with “Eating Disorder” being displayed or highlighted in the diagnosis summary portion 335 (as described above).

In some embodiments, referring to the non-limiting example of FIG. 3B, the diagnosis summary table portion 340 might display a table depicting the probability (in percentage values) of the likelihood the patient is diagnosed with the following eating disorders or no eating disorder: anorexia nervosa (“AN”; in this case, 75%); bulimia nervosa (“BN”; in this case, 10%), other specified feeding or eating disorder (“OSFED”; in this case, 8%), binge-eating disorder (“BED”; in this case, 4%), obesity (“OB”; in this case, 2%), or no eating disorder (“No ED”; in this case, 1%), or the like. In the non-limiting example of FIG. 3, as the probability of the patient having AN is an overwhelming 75%, AN and 75% are highlighted in boldface, with AN being displayed or highlighted in the diagnosis summary portion 335 (as described above).

FIGS. 3A and 3B are not mutually exclusive and may be examples of views of the display in different selectable modes, the first mode being a general diagnosis and probability of whether or not the patient is likely to have at least one eating disorder (as shown in FIG. 3A), while the second mode is a specific breakdown of the diagnoses and probabilities of the particular types of eating disorders (e.g., AN, BN, OSFED, BED, or OB, etc.) (as shown in FIG. 3B). Alternatively, FIGS. 3A and 3B may be alternative implementations. Although not expressly shown in FIG. 3, the details tab 325 might provide detailed information regarding the diagnosis. In some embodiments,

In some cases, the main portion 315 might include one or more (virtual or user interface) buttons 345, including, but not limited to, a new questionnaire button 345a (which, when actuated or clicked, presents the patient with a new questionnaire list a set of questions each having closed-ended answer options), a show all button 345b (which, when actuated or clicked, presents all available relevant information associated with the patient or presents a list of results for a plurality of patients), and a delete button 345c (which, when actuated or clicked, deletes the presented results), or the like. In some instances, the user interface or app might further comprise a footer portion 350, which might include, without limitation, icons or buttons for flipping through various results associated with the particular patient, icons or buttons for flipping through various results associated with each of a plurality of the patients, icons or buttons for adding or deleting diagnosis results, icons or buttons for sorting results alphabetically or reverse alphabetically by patient name, by ID number, by age, by eating disorder diagnosed, or the like.

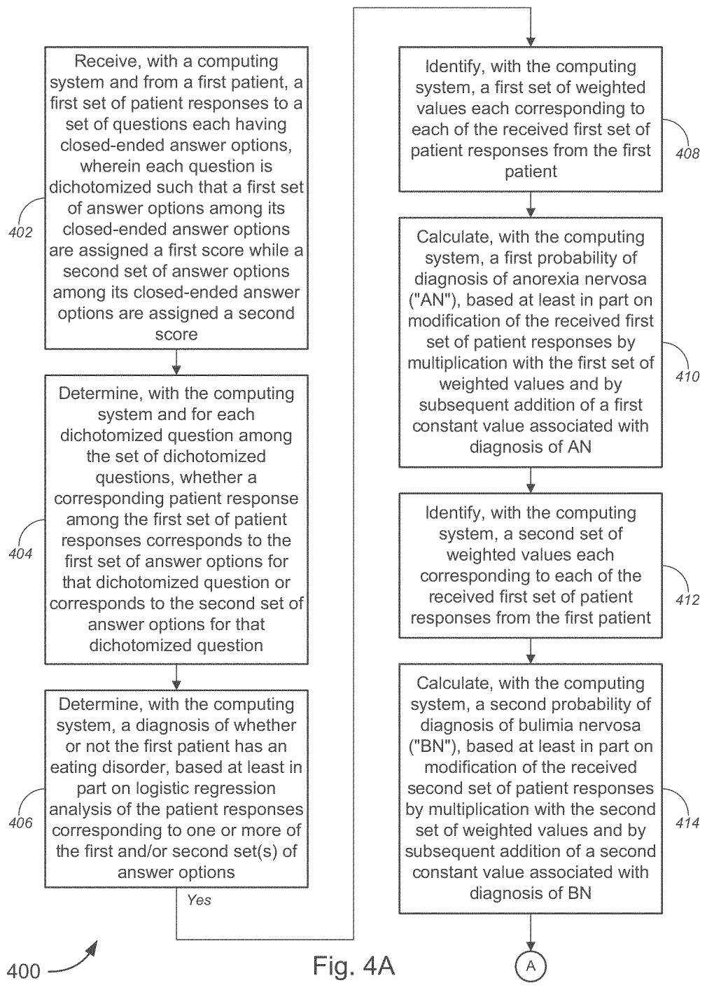

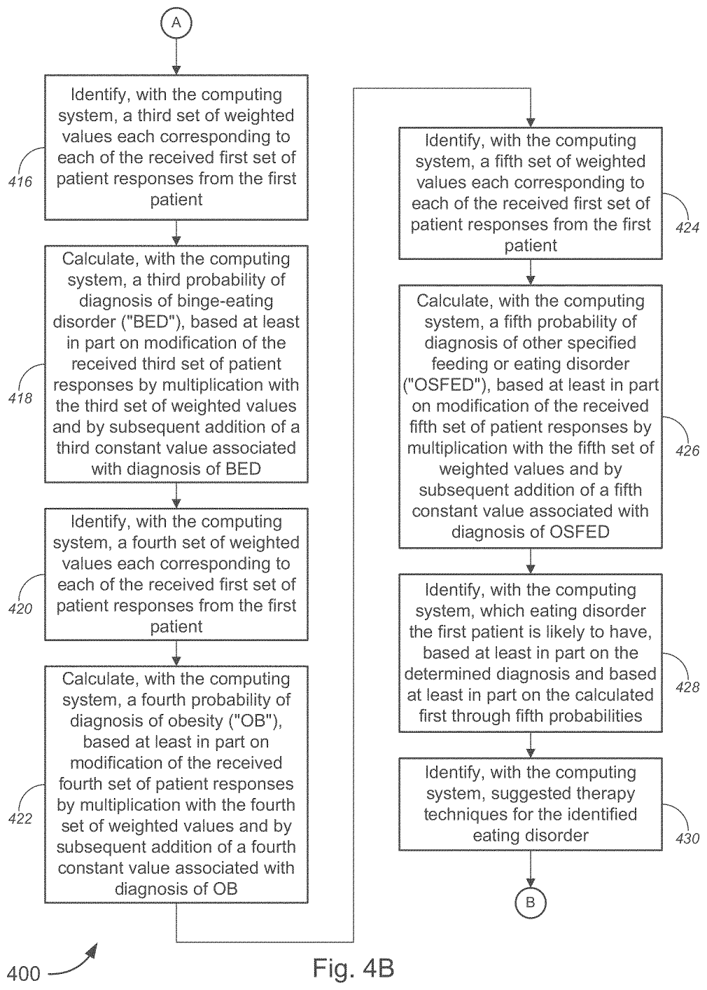

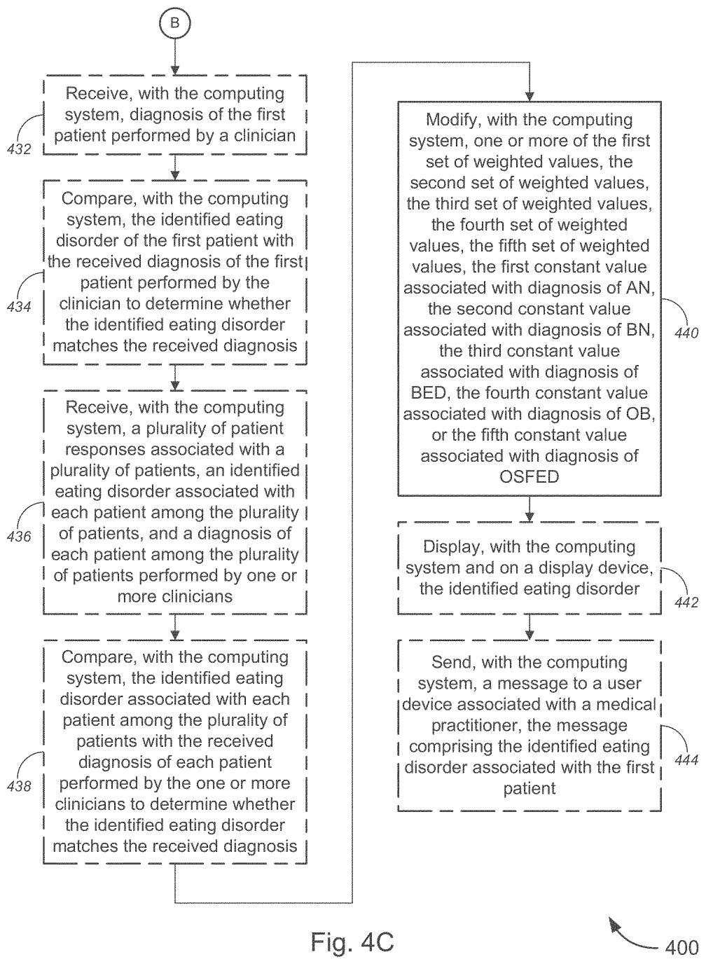

FIGS. 4A-4C (collectively, “FIG. 4”) are flow diagrams illustrating a method for implementing eating disorder diagnosis, in accordance with various embodiments. Method 400 of FIG. 4A continues onto FIG. 4B following the circular marker denoted, “A,” and continues from FIG. 4B onto FIG. 4C following the circular marker denoted, “B.”

While the techniques and procedures are depicted and/or described in a certain order for purposes of illustration, it should be appreciated that certain procedures may be reordered and/or omitted within the scope of various embodiments. Moreover, while the method 400 illustrated by FIG. 4 can be implemented by or with (and, in some cases, are described below with respect to) the systems, examples, or embodiments 100, 200-200″, and 300 of FIGS. 1, 2, and 3, respectively (or components thereof), such methods may also be implemented using any suitable hardware (or software) implementation. Similarly, while each of the systems, examples, or embodiments 100, 200-200″, and 300 of FIGS. 1, 2, and 3, respectively (or components thereof), can operate according to the method 400 illustrated by FIG. 4 (e.g., by executing instructions embodied on a computer readable medium), the systems, examples, or embodiments 100, 200-200″, and 300 of FIGS. 1, 2, and 3 can each also operate according to other modes of operation and/or perform other suitable procedures.

In the non-limiting embodiment of FIG. 4A, method 400, at block 402, might comprise receiving, with a computing system and from a first patient, a first set of patient responses to a set of questions each having closed-ended answer options, wherein each question is dichotomized such that a first set of answer options among its closed-ended answer options are assigned a first score while a second set of answer options among its closed-ended answer options are assigned a second score.

In some embodiments, the set of questions might include, without limitation, a first category of questions, a second category of questions, and a third category of questions, or the like. The first category of questions might comprise questions regarding conditions including, but not limited to, at least one of body-mass index (“BMI”), weight loss during the previous year, or self-induced vomiting, and/or the like. The second category of questions might comprise questions regarding behavior including, without limitation, at least one of eating patterns, dieting, weighing one's self, isolation from friends and family, or avoiding activities, and/or the like. The third category of questions might comprise questions regarding thoughts including, but not limited to, at least one of being afraid of losing control over eating, thoughts about food, believing one's self to be fat when others call one too thin, or reaction to weight gain, and/or the like. The first set of weighted values might be differently defined based on differences among the first category of questions, the second category of questions, and the third category of questions.

At block 404, method 400 might comprise determining, with the computing system and for each dichotomized question among the set of dichotomized questions, whether a corresponding patient response among the first set of patient responses corresponds to the first set of answer options for that dichotomized question or corresponds to the second set of answer options for that dichotomized question. Method 400 might, at block 406, comprise determining, with the computing system, a diagnosis of whether or not the first patient has an eating disorder, based at least in part on logistic regression analysis of one or more of the patient responses among the first set of patient responses corresponding to the first set of answer options for the set of dichotomized questions or the patient responses among the first set of patient responses corresponding to the second set of answer options for the set of dichotomized questions and/or based at least in part on logistic regression analysis of one or more of the patient responses among the first set of patient responses corresponding to the first set of answer options that are assigned the first score or the patient responses among the first set of patient responses corresponding to the second set of answer options that are assigned the second score.

Method 400 might further comprise, based on the determination that the first patient likely has an eating disorder, performing one or more of the following: identifying, with the computing system, a first set of weighted values each corresponding to each of the received first set of patient responses from the first patient (block 408); and calculating, with the computing system, a first probability of diagnosis of anorexia nervosa (“AN”), based at least in part on modification of the received first set of patient responses by multiplication with the first set of weighted values and by subsequent addition of a first constant value associated with diagnosis of AN (block 410).

Similarly, method 400 might further comprise identifying, with the computing system, a second set of weighted values each corresponding to each of the received first set of patient responses from the first patient (block 412); and calculating, with the computing system, a second probability of diagnosis of bulimia nervosa (“BN”), based at least in part on modification of the received second set of patient responses by multiplication with the second set of weighted values and by subsequent addition of a second constant value associated with diagnosis of BN (block 414).

Method 400 might continue onto the process at block 416 in FIG. 4B following the circular marker denoted, “A.” At block 416 in FIG. 4B (following the circular marker denoted, “A”), method 400 might comprise identifying, with the computing system, a third set of weighted values each corresponding to each of the received first set of patient responses from the first patient. At block 418, method 400 might comprise calculating, with the computing system, a third probability of diagnosis of binge-eating disorder (“BED”), based at least in part on modification of the received third set of patient responses by multiplication with the third set of weighted values and by subsequent addition of a third constant value associated with diagnosis of BED.

Similarly, method 400 might further comprise identifying, with the computing system, a fourth set of weighted values each corresponding to each of the received first set of patient responses from the first patient (block 420); and calculating, with the computing system, a fourth probability of diagnosis of obesity (“OB”), based at least in part on modification of the received fourth set of patient responses by multiplication with the fourth set of weighted values and by subsequent addition of a fourth constant value associated with diagnosis of OB (block 422).

Method 400 might further comprise identifying, with the computing system, a fifth set of weighted values each corresponding to each of the received first set of patient responses from the first patient (block 424); and calculating, with the computing system, a fifth probability of diagnosis of other specified feeding or eating disorder (“OSFED”), based at least in part on modification of the received fifth set of patient responses by multiplication with the fifth set of weighted values and by subsequent addition of a fifth constant value associated with diagnosis of OSFED (block 426).

At block 428, method 400 might comprise identifying, with the computing system, which eating disorder the first patient is likely to have, based at least in part on the determined diagnosis and based at least in part on the calculated first through fifth probabilities. Method 400 might further comprise, at block 430, identifying, with the computing system, suggested therapy techniques for the identified eating disorder. Method 400 might continue onto the process at optional block 432 in FIG. 4C following the circular marker denoted, “B.”

At optional block 432 in FIG. 4C (following the circular marker denoted, “B”), method 400 might comprise receiving, with the computing system, diagnosis of the first patient performed by a clinician. Method 400 might further comprise, at optional block 434, comparing, with the computing system, the identified eating disorder of the first patient with the received diagnosis of the first patient performed by the clinician to determine whether the identified eating disorder matches the received diagnosis. Method 400, at optional block 436, might comprise receiving, with the computing system, a plurality of patient responses associated with a plurality of patients, an identified eating disorder associated with each patient among the plurality of patients, and a diagnosis of each patient among the plurality of patients performed by one or more clinicians. Method 400 might further comprise, at optional block 438, comparing, with the computing system, the identified eating disorder associated with each patient among the plurality of patients with the received diagnosis of each patient performed by the one or more clinicians to determine whether the identified eating disorder matches the received diagnosis. In some cases, the processes at optional blocks 432-438 might be performed during the development phase, during periodic or occasional training periods, or for periodic or occasional enhancement of the autonomous diagnosis functionalities, or the like, to validate the diagnostics made by the algorithm.

Method 400 might further comprise modifying, with the computing system, one or more of the first set of weighted values, the second set of weighted values, the third set of weighted values, the fourth set of weighted values, the fifth set of weighted values, the first constant value associated with diagnosis of AN, the second constant value associated with diagnosis of BN, the third constant value associated with diagnosis of BED, the fourth constant value associated with diagnosis of OB, or the fifth constant value associated with diagnosis of OSFED (block 440). In some embodiments, modifying the one or more of the first through fifth sets of weighted values and/or the first through fifth constant values might be based at least in part on one or more of the comparison of the identified eating disorder of the first patient with the received diagnosis of the first patient performed by the clinician, the comparison of the identified eating disorder associated with each patient among the plurality of patients with the received diagnosis of each patient performed by the one or more clinicians, the received first set of patient responses, and/or the received plurality of patient responses associated with the plurality of patients. In other words, as more and more patients are correctly or incorrectly diagnosed or classified in terms of eating disorders that the patients might have, the computing system may be further trained to more accurately diagnose or classify future eating disorders in future patients. According to some embodiments, to avoid training biases, the weights or coefficients and constants may be reset every N number of patients, where N is a predetermined number of patients.

At optional block 442, method 400 might comprise displaying, with the computing system and on a display device, the identified eating disorder. In some embodiments, the display device might include, without limitation, one of a tablet computer, a smart phone, a mobile phone, a laptop computer, a desktop computer, or a monitor, and/or the like. In some cases, receiving the first set of patient responses might comprise receiving, with the computing system and from a user device that receives input from the first patient, the first set of patient responses to the set of questions. In some instances, displaying the identified eating disorder might comprise displaying, with the computing system, the identified eating disorder in a software application (“app”) running on the display device.

Method 400, at optional block 444, might comprise sending, with the computing system, a message to a user device associated with a medical practitioner, the message comprising the identified eating disorder associated with the first patient. According to some embodiments, the medical practitioner might include, but is not limited to one of a general medical practitioner, a primary care physician, a psychiatrist, a clinician, or a nurse, and/or the like. In some instances, the user device associated with the medical practitioner might include, without limitation, one of a tablet computer, a smart phone, a mobile phone, a laptop computer, or a desktop computer, and/or the like. In some cases, the message might further comprise suggested therapy techniques associated with the identified eating disorder associated with the first patient.

Exemplary System and Hardware Implementation

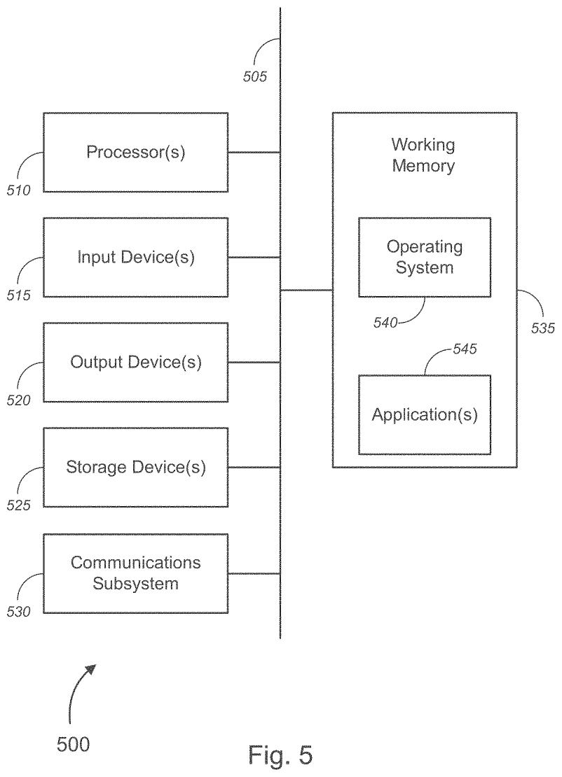

FIG. 5 is a block diagram illustrating an exemplary computer or system hardware architecture, in accordance with various embodiments. FIG. 5 provides a schematic illustration of one embodiment of a computer system 500 of the service provider system hardware that can perform the methods provided by various other embodiments, as described herein, and/or can perform the functions of computer or hardware system (i.e., computing systems 105a and 105b, user devices 115 and 300 used by a patient, user device(s) 125 used by healthcare professionals, and medical server(s) 140, etc.), as described above. It should be noted that FIG. 5 is meant only to provide a generalized illustration of various components, of which one or more (or none) of each may be utilized as appropriate. FIG. 5, therefore, broadly illustrates how individual system elements may be implemented in a relatively separated or relatively more integrated manner.

The computer or hardware system 500—which might represent an embodiment of the computer or hardware system (i.e., computing systems 105a and 105b, user devices 115 and 300 used by a patient, user device(s) 125 used by healthcare professionals, and medical server(s) 140, etc.), described above with respect to FIGS. 1-4—is shown comprising hardware elements that can be electrically coupled via a bus 505 (or may otherwise be in communication, as appropriate). The hardware elements may include one or more processors 510, including, without limitation, one or more general-purpose processors and/or one or more special-purpose processors (such as microprocessors, digital signal processing chips, graphics acceleration processors, and/or the like); one or more input devices 515, which can include, without limitation, a mouse, a keyboard, and/or the like; and one or more output devices 520, which can include, without limitation, a display device, a printer, and/or the like.

The computer or hardware system 500 may further include (and/or be in communication with) one or more storage devices 525, which can comprise, without limitation, local and/or network accessible storage, and/or can include, without limitation, a disk drive, a drive array, an optical storage device, solid-state storage device such as a random access memory (“RAM”) and/or a read-only memory (“ROM”), which can be programmable, flash-updateable, and/or the like. Such storage devices may be configured to implement any appropriate data stores, including, without limitation, various file systems, database structures, and/or the like.

The computer or hardware system 500 might also include a communications subsystem 530, which can include, without limitation, a modem, a network card (wireless or wired), an infra-red communication device, a wireless communication device and/or chipset (such as a Bluetooth™ device, an 802.11 device, a WiFi device, a WiMax device, a WWAN device, cellular communication facilities, etc.), and/or the like. The communications subsystem 530 may permit data to be exchanged with a network (such as the network described below, to name one example), with other computer or hardware systems, and/or with any other devices described herein. In many embodiments, the computer or hardware system 500 will further comprise a working memory 535, which can include a RAM or ROM device, as described above.

The computer or hardware system 500 also may comprise software elements, shown as being currently located within the working memory 535, including an operating system 540, device drivers, executable libraries, and/or other code, such as one or more application programs 545, which may comprise computer programs provided by various embodiments (including, without limitation, hypervisors, VMs, and the like), and/or may be designed to implement methods, and/or configure systems, provided by other embodiments, as described herein. Merely by way of example, one or more procedures described with respect to the method(s) discussed above might be implemented as code and/or instructions executable by a computer (and/or a processor within a computer); in an aspect, then, such code and/or instructions can be used to configure and/or adapt a general purpose computer (or other device) to perform one or more operations in accordance with the described methods.

A set of these instructions and/or code might be encoded and/or stored on a non-transitory computer readable storage medium, such as the storage device(s) 525 described above. In some cases, the storage medium might be incorporated within a computer system, such as the system 500. In other embodiments, the storage medium might be separate from a computer system (i.e., a removable medium, such as a compact disc, etc.), and/or provided in an installation package, such that the storage medium can be used to program, configure, and/or adapt a general purpose computer with the instructions/code stored thereon. These instructions might take the form of executable code, which is executable by the computer or hardware system 500 and/or might take the form of source and/or installable code, which, upon compilation and/or installation on the computer or hardware system 500 (e.g., using any of a variety of generally available compilers, installation programs, compression/decompression utilities, etc.) then takes the form of executable code.

It will be apparent to those skilled in the art that substantial variations may be made in accordance with specific requirements. For example, customized hardware (such as programmable logic controllers, field-programmable gate arrays, application-specific integrated circuits, and/or the like) might also be used, and/or particular elements might be implemented in hardware, software (including portable software, such as applets, etc.), or both. Further, connection to other computing devices such as network input/output devices may be employed.

As mentioned above, in one aspect, some embodiments may employ a computer or hardware system (such as the computer or hardware system 500) to perform methods in accordance with various embodiments of the invention. According to a set of embodiments, some or all of the procedures of such methods are performed by the computer or hardware system 500 in response to processor 510 executing one or more sequences of one or more instructions (which might be incorporated into the operating system 540 and/or other code, such as an application program 545) contained in the working memory 535. Such instructions may be read into the working memory 535 from another computer readable medium, such as one or more of the storage device(s) 525. Merely by way of example, execution of the sequences of instructions contained in the working memory 535 might cause the processor(s) 510 to perform one or more procedures of the methods described herein.

The terms “machine readable medium” and “computer readable medium,” as used herein, refer to any medium that participates in providing data that causes a machine to operate in a specific fashion. In an embodiment implemented using the computer or hardware system 500, various computer readable media might be involved in providing instructions/code to processor(s) 510 for execution and/or might be used to store and/or carry such instructions/code (e.g., as signals). In many implementations, a computer readable medium is a non-transitory, physical, and/or tangible storage medium. In some embodiments, a computer readable medium may take many forms, including, but not limited to, non-volatile media, volatile media, or the like. Non-volatile media includes, for example, optical and/or magnetic disks, such as the storage device(s) 525. Volatile media includes, without limitation, dynamic memory, such as the working memory 535. In some alternative embodiments, a computer readable medium may take the form of transmission media, which includes, without limitation, coaxial cables, copper wire, and fiber optics, including the wires that comprise the bus 505, as well as the various components of the communication subsystem 530 (and/or the media by which the communications subsystem 530 provides communication with other devices). In an alternative set of embodiments, transmission media can also take the form of waves (including without limitation radio, acoustic, and/or light waves, such as those generated during radio-wave and infra-red data communications).

Common forms of physical and/or tangible computer readable media include, for example, a floppy disk, a flexible disk, a hard disk, magnetic tape, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave as described hereinafter, or any other medium from which a computer can read instructions and/or code.

Various forms of computer readable media may be involved in carrying one or more sequences of one or more instructions to the processor(s) 510 for execution. Merely by way of example, the instructions may initially be carried on a magnetic disk and/or optical disc of a remote computer. A remote computer might load the instructions into its dynamic memory and send the instructions as signals over a transmission medium to be received and/or executed by the computer or hardware system 500. These signals, which might be in the form of electromagnetic signals, acoustic signals, optical signals, and/or the like, are all examples of carrier waves on which instructions can be encoded, in accordance with various embodiments of the invention.

The communications subsystem 530 (and/or components thereof) generally will receive the signals, and the bus 505 then might carry the signals (and/or the data, instructions, etc. carried by the signals) to the working memory 535, from which the processor(s) 505 retrieves and executes the instructions. The instructions received by the working memory 535 may optionally be stored on a storage device 525 either before or after execution by the processor(s) 510.

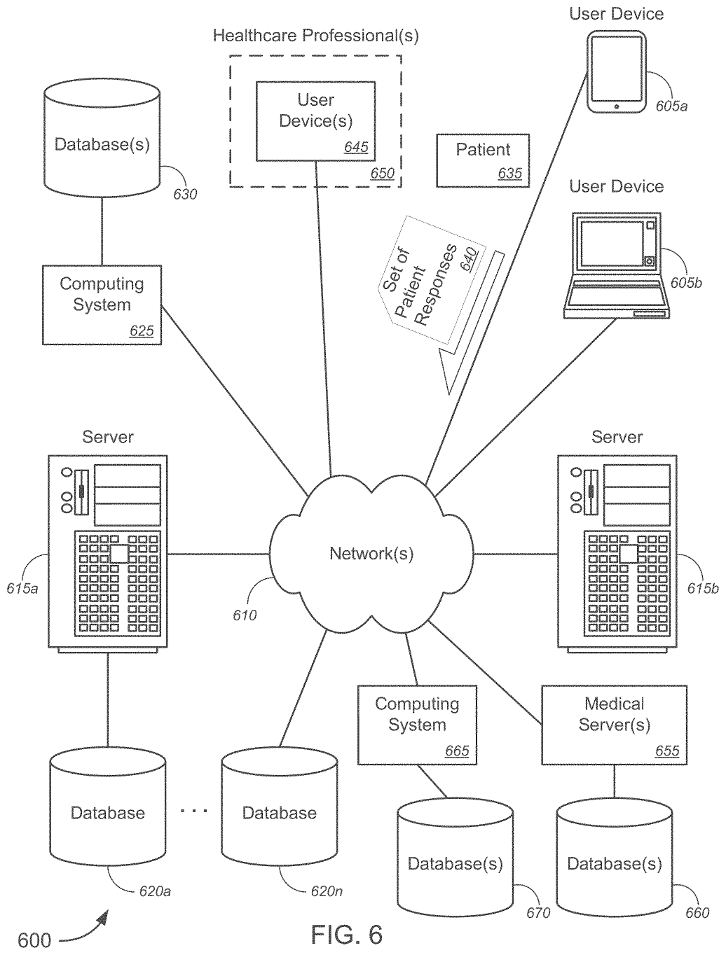

As noted above, a set of embodiments comprises methods and systems for implementing eating disorder diagnosis, and, more particularly, to methods, systems, and apparatuses for implementing eating disorder diagnosis based on analysis of patient responses to a set of questions having closed-ended answer options. FIG. 6 illustrates a schematic diagram of a system 600 that can be used in accordance with one set of embodiments. The system 600 can include one or more user computers, user devices, or customer devices 605. A user computer, user device, or customer device 605 can be a general purpose personal computer (including, merely by way of example, desktop computers, tablet computers, laptop computers, handheld computers, and the like, running any appropriate operating system, several of which are available from vendors such as Apple, Microsoft Corp., and the like), cloud computing devices, a server(s), and/or a workstation computer(s) running any of a variety of commercially-available UNIX™ or UNIX-like operating systems. A user computer, user device, or customer device 605 can also have any of a variety of applications, including one or more applications configured to perform methods provided by various embodiments (as described above, for example), as well as one or more office applications, database client and/or server applications, and/or web browser applications. Alternatively, a user computer, user device, or customer device 605 can be any other electronic device, such as a thin-client computer, Internet-enabled mobile telephone, and/or personal digital assistant, capable of communicating via a network (e.g., the network(s) 610 described below) and/or of displaying and navigating web pages or other types of electronic documents. Although the exemplary system 600 is shown with two user computers, user devices, or customer devices 605, any number of user computers, user devices, or customer devices can be supported.

Certain embodiments operate in a networked environment, which can include a network(s) 610. The network(s) 610 can be any type of network familiar to those skilled in the art that can support data communications using any of a variety of commercially-available (and/or free or proprietary) protocols, including, without limitation, TCP/IP, SNA™ IPX™ AppleTalk™, and the like. Merely by way of example, the network(s) 610 (similar to network(s) 135 FIG. 1, or the like) can each include a local area network (“LAN”), including, without limitation, a fiber network, an Ethernet network, a Token-Ring™ network, and/or the like; a wide-area network (“WAN”); a wireless wide area network (“WWAN”); a virtual network, such as a virtual private network (“VPN”); the Internet; an intranet; an extranet; a public switched telephone network (“PSTN”); an infra-red network; a wireless network, including, without limitation, a network operating under any of the IEEE 802.11 suite of protocols, the Bluetooth™ protocol known in the art, and/or any other wireless protocol; and/or any combination of these and/or other networks. In a particular embodiment, the network might include an access network of the service provider (e.g., an Internet service provider (“ISP”)). In another embodiment, the network might include a core network of the service provider, and/or the Internet.

Embodiments can also include one or more server computers 615. Each of the server computers 615 may be configured with an operating system, including, without limitation, any of those discussed above, as well as any commercially (or freely) available server operating systems. Each of the servers 615 may also be running one or more applications, which can be configured to provide services to one or more clients 605 and/or other servers 615.

Merely by way of example, one of the servers 615 might be a data server, a web server, a cloud computing device(s), or the like, as described above. The data server might include (or be in communication with) a web server, which can be used, merely by way of example, to process requests for web pages or other electronic documents from user computers 605. The web server can also run a variety of server applications, including HTTP servers, FTP servers, CGI servers, database servers, Java servers, and the like. In some embodiments of the invention, the web server may be configured to serve web pages that can be operated within a web browser on one or more of the user computers 605 to perform methods of the invention.

The server computers 615, in some embodiments, might include one or more application servers, which can be configured with one or more applications accessible by a client running on one or more of the client computers 605 and/or other servers 615. Merely by way of example, the server(s) 615 can be one or more general purpose computers capable of executing programs or scripts in response to the user computers 605 and/or other servers 615, including, without limitation, web applications (which might, in some cases, be configured to perform methods provided by various embodiments). Merely by way of example, a web application can be implemented as one or more scripts or programs written in any suitable programming language, such as Java™, C, C#™ or C++, and/or any scripting language, such as Perl, Python, or TCL, as well as combinations of any programming and/or scripting languages. The application server(s) can also include database servers, including, without limitation, those commercially available from Oracle™, Microsoft™, Sybase™ IBM™, and the like, which can process requests from clients (including, depending on the configuration, dedicated database clients, API clients, web browsers, etc.) running on a user computer, user device, or customer device 605 and/or another server 615. In some embodiments, an application server can perform one or more of the processes for implementing eating disorder diagnosis, and, more particularly, to methods, systems, and apparatuses for implementing eating disorder diagnosis based on analysis of patient responses to a set of questions having closed-ended answer options, as described in detail above. Data provided by an application server may be formatted as one or more web pages (comprising HTML, JavaScript, etc., for example) and/or may be forwarded to a user computer 605 via a web server (as described above, for example). Similarly, a web server might receive web page requests and/or input data from a user computer 605 and/or forward the web page requests and/or input data to an application server. In some cases, a web server may be integrated with an application server.

In accordance with further embodiments, one or more servers 615 can function as a file server and/or can include one or more of the files (e.g., application code, data files, etc.) necessary to implement various disclosed methods, incorporated by an application running on a user computer 605 and/or another server 615. Alternatively, as those skilled in the art will appreciate, a file server can include all necessary files, allowing such an application to be invoked remotely by a user computer, user device, or customer device 605 and/or server 615.

It should be noted that the functions described with respect to various servers herein (e.g., application server, database server, web server, file server, etc.) can be performed by a single server and/or a plurality of specialized servers, depending on implementation-specific needs and parameters.

In certain embodiments, the system can include one or more databases 620a-620n (collectively, “databases 620”). The location of each of the databases 620 is discretionary: merely by way of example, a database 620a might reside on a storage medium local to (and/or resident in) a server 615a (and/or a user computer, user device, or customer device 605). Alternatively, a database 620n can be remote from any or all of the computers 605, 615, so long as it can be in communication (e.g., via the network 610) with one or more of these. In a particular set of embodiments, a database 620 can reside in a storage-area network (“SAN”) familiar to those skilled in the art. (Likewise, any necessary files for performing the functions attributed to the computers 605, 615 can be stored locally on the respective computer and/or remotely, as appropriate.) In one set of embodiments, the database 620 can be a relational database, such as an Oracle database, that is adapted to store, update, and retrieve data in response to SQL-formatted commands. The database might be controlled and/or maintained by a database server, as described above, for example.

According to some embodiments, system 600 might further comprise a computing system 625 and corresponding database(s) 630 (similar to computing system 105a and corresponding database(s) 110a of FIG. 1, or the like). System 600 might further comprise a patient 635 (similar to patient 120 of FIG. 1, or the like) that use user device 605a or 605b to enter and send a set of patient response 640 (similar to set of patient responses 155 of FIG. 1, or the like) to a set of questions (not shown in FIG. 6; similar to set of questions 150 of FIG. 1, or the like), via network(s) 610, to computing system 625 and/or user device(s) 645 used by healthcare professional(s) 650. In some embodiments, system 600 might further comprise medical server(s) 655 and corresponding database(s) 660 (similar to medical server(s) 140 and corresponding database(s) 145 of FIG. 1, or the like), remote computing system 665 and corresponding database(s) 670 (similar to computing system 105b and corresponding database(s) 110b of FIG. 1, or the like), or the like.

In operation, computing system 625, computing system 665, user device(s) 605a or 605b, user device(s) 645, and/or medical server(s) 655 (collectively, “computing system”) might receive, from a first patient 635 (e.g., via the user device(s) 605a or 605b or via a user interface device (such as a voice input, a touch input, a key or button input, or the like) of the user device(s) 605a or 605b, or the like), a first set of patient responses 640 to a set of questions each having closed-ended answer options. Each question might be dichotomized such that a first set of answer options among its closed-ended answer options are assigned a first score while a second set of answer options among its closed-ended answer options are assigned a second score.

In some embodiments, the set of questions might include, without limitation, a first category of questions, a second category of questions, and a third category of questions, or the like. The first category of questions might comprise questions regarding conditions including, but not limited to, at least one of body-mass index (“BMI”), weight loss during the previous year, or self-induced vomiting, and/or the like. The second category of questions might comprise questions regarding behavior including, without limitation, at least one of eating patterns, dieting, weighing one's self, isolation from friends and family, or avoiding activities, and/or the like. The third category of questions might comprise questions regarding thoughts including, but not limited to, at least one of being afraid of losing control over eating, thoughts about food, believing one's self to be fat when others call one too thin, or reaction to weight gain, and/or the like. The first set of weighted values might be differently defined based on differences among the first category of questions, the second category of questions, and the third category of questions. The computing system might determine, for each dichotomized question among the set of dichotomized questions, whether a corresponding patient response among the first set of patient responses corresponds to the first set of answer options for that dichotomized question or corresponds to the second set of answer options for that dichotomized question, wherein the first set of answer options for the set of dichotomized questions is indicative of likelihood of patients selecting such answer options having an eating disorder, and wherein the second set of answer options for the set of dichotomized questions is indicative of likelihood of patients selecting such answer options not having an eating disorder.

The computing system might determine a diagnosis of whether or not the first patient has an eating disorder, based at least in part on logistic regression analysis of one or more of the patient responses among the first set of patient responses corresponding to the first set of answer options for the set of dichotomized questions or the patient responses among the first set of patient responses corresponding to the second set of answer options for the set of dichotomized questions. Based on a determination that the first patient likely has an eating disorder, the computing system might perform one or more of the following: (a) identify a first set of weighted values each corresponding to each of the received first set of patient responses from the first patient, and calculate a first probability of diagnosis of a first eating disorder, based at least in part on modification of the received first set of patient responses by multiplication with the first set of weighted values and by subsequent addition of a first constant value associated with diagnosis of the first eating disorder; (b) identify a second set of weighted values each corresponding to each of the received first set of patient responses from the first patient, and calculate a second probability of diagnosis of a second eating disorder, based at least in part on modification of the received second set of patient responses by multiplication with the second set of weighted values and by subsequent addition of a second constant value associated with diagnosis of the second eating disorder; (c) identify a third set of weighted values each corresponding to each of the received first set of patient responses from the first patient, and calculate a third probability of diagnosis of a third eating disorder, based at least in part on modification of the received third set of patient responses by multiplication with the third set of weighted values and by subsequent addition of a third constant value associated with diagnosis of the third eating disorder; (d) identify a fourth set of weighted values each corresponding to each of the received first set of patient responses from the first patient, and calculate a fourth probability of diagnosis of a fourth eating disorder, based at least in part on modification of the received fourth set of patient responses by multiplication with the fourth set of weighted values and by subsequent addition of a fourth constant value associated with diagnosis of the fourth eating disorder; and/or (e) identify a fifth set of weighted values each corresponding to each of the received first set of patient responses from the first patient, and might calculate a fifth probability of diagnosis of a fifth eating disorder, based at least in part on modification of the received fifth set of patient responses by multiplication with the fifth set of weighted values and by subsequent addition of a fifth constant value associated with diagnosis of the fifth eating disorder; and/or the like. In some embodiments, the first eating disorder, the second eating disorder, the third eating disorder, the fourth eating disorder, and the fifth eating disorder might each comprise one of anorexia nervosa (“AN”), bulimia nervosa (“BN”), binge-eating disorder (“BED”), obesity (“OB”), or other specified feeding or eating disorder (“OSFED”), and/or the like.

The computing system might identify which eating disorder the first patient is likely to have, based at least in part on the determined diagnosis and based at least in part on the calculated first through fifth probabilities. In some embodiments, the computing system might identify suggested therapy techniques for the identified eating disorder. In some cases, the computing system might display, on a display device (e.g., on display screen(s) 605a or 605ba of user device(s) 605a or 605b, or the like), the identified eating disorder and/or the identified suggested therapy techniques for the identified eating disorder. In some cases, displaying the identified eating disorder might comprise displaying, with the computing system, the identified eating disorder in a software application (“app”) running on the display device. In some embodiments, the computing system might send a message to user device(s) 645 associated with the clinician or healthcare professional 650, the message comprising the identified eating disorder associated with the first patient 635. In some instances, the message might further comprise the identified suggested therapy techniques associated with the identified eating disorder associated with the first patient.

According to some embodiments, such as during the development phase, during periodic or occasional training periods, or for periodic or occasional enhancement of the autonomous diagnosis functionalities, and/or the like, the computing system might receive diagnosis of the first patient 635 performed by a clinician or healthcare professional 650 among the one or more healthcare professionals 650. The computing system might compare the identified eating disorder of the first patient with the received diagnosis of the first patient performed by the clinician or healthcare professional 650 to determine whether the identified eating disorder matches the received diagnosis. In some cases, the computing system might further display, on the display device (e.g., on display screen(s) 605a or 605ba of user device(s) 605a or 605b, or the like), the received diagnosis of the first patient 635 performed by the clinician or healthcare professional 650. In some embodiments, the computing system might receive a plurality of patient responses associated with a plurality of patients, an identified eating disorder associated with each patient among the plurality of patients, and a diagnosis of each patient among the plurality of patients performed by one or more clinicians or healthcare professionals 650. The computing system might compare the identified eating disorder associated with each patient among the plurality of patients with the received diagnosis of each patient performed by the one or more clinicians or healthcare professionals 650 to determine whether the identified eating disorder matches the received diagnosis. Based on the comparison, the computing system might analyze the plurality of sets of patient responses associated with the plurality of patients, the identified eating disorder associated with each patient among the plurality of patients, the diagnosis of each patient among the plurality of patients performed by the one or more clinicians or healthcare professionals 650, and one or more of the first through fifth set of weighted values to determine whether one or more of the first set of weighted values, the second set of weighted values, the third set of weighted values, the fourth set of weighted values, the fifth set of weighted values, the first constant value associated with diagnosis of the first eating disorder, the second constant value associated with diagnosis of the second eating disorder, the third constant value associated with diagnosis of the third eating disorder, the fourth constant value associated with diagnosis of the fourth eating disorder, or the fifth constant value associated with diagnosis of the fifth eating disorder are optimal or should be updated or modified, and/or the like.

The computing system might modify one or more of the first set of weighted values, the second set of weighted values, the third set of weighted values, the fourth set of weighted values, the fifth set of weighted values, the first constant value associated with diagnosis of the first eating disorder, the second constant value associated with diagnosis of the second eating disorder, the third constant value associated with diagnosis of the third eating disorder, the fourth constant value associated with diagnosis of the fourth eating disorder, or the fifth constant value associated with diagnosis of the fifth eating disorder, based at least in part on one or more of the comparison of the identified eating disorder associated with each patient among the plurality of patients (including the first patient 635) with the received diagnosis of each patient performed by the one or more clinicians or healthcare professionals 650 and/or the received plurality of patient responses associated with the plurality of patients.

These and other functions of the system 600 (and its components) are described in greater detail above with respect to FIGS. 1-4.

While certain features and aspects have been described with respect to exemplary embodiments, one skilled in the art will recognize that numerous modifications are possible. For example, the methods and processes described herein may be implemented using hardware components, software components, and/or any combination thereof. Further, while various methods and processes described herein may be described with respect to particular structural and/or functional components for ease of description, methods provided by various embodiments are not limited to any particular structural and/or functional architecture but instead can be implemented on any suitable hardware, firmware and/or software configuration. Similarly, while certain functionality is ascribed to certain system components, unless the context dictates otherwise, this functionality can be distributed among various other system components in accordance with the several embodiments.

Moreover, while the procedures of the methods and processes described herein are described in a particular order for ease of description, unless the context dictates otherwise, various procedures may be reordered, added, and/or omitted in accordance with various embodiments. Moreover, the procedures described with respect to one method or process may be incorporated within other described methods or processes; likewise, system components described according to a particular structural architecture and/or with respect to one system may be organized in alternative structural architectures and/or incorporated within other described systems. Hence, while various embodiments are described with—or without—certain features for ease of description and to illustrate exemplary aspects of those embodiments, the various components and/or features described herein with respect to a particular embodiment can be substituted, added and/or subtracted from among other described embodiments, unless the context dictates otherwise. Consequently, although several exemplary embodiments are described above, it will be appreciated that the invention is intended to cover all modifications and equivalents within the scope of the following claims.

What is claimed is:

1. A method, comprising:
receiving, with a computing system and from a first patient, a first set of patient responses to a set of questions each having closed-ended answer options, wherein each question is dichotomized such that a first set of answer options among its closed-ended answer options are assigned a first score while a second set of answer options among its closed-ended answer options are assigned a second score;
determining, with the computing system and for each dichotomized question among the set of dichotomized questions, whether a corresponding patient response among the first set of patient responses corresponds to the first set of answer options for that dichotomized question or corresponds to the second set of answer options for that dichotomized question, wherein the first set of answer options for the set of dichotomized questions is indicative of likelihood of patients selecting such answer options having an eating disorder, and wherein the second set of answer options for the set of dichotomized questions is indicative of likelihood of patients selecting such answer options not having an eating disorder;
determining, with the computing system, a diagnosis of whether or not the first patient has an eating disorder, based at least in part on logistic regression analysis of one or more of the patient responses among the first set of patient responses corresponding to the first set of answer options for the set of dichotomized questions or the patient responses among the first set of patient responses corresponding to the second set of answer options for the set of dichotomized questions; and
based on a determination that the first patient likely has an eating disorder, performing one or more of the following:
identifying, with the computing system, a first set of weighted values each corresponding to each of the received first set of patient responses from the first patient;
calculating, with the computing system, a first probability of diagnosis of anorexia nervosa (“AN”), based at least in part on modification of the received first set of patient responses by multiplication with the first set of weighted values and by subsequent addition of a first constant value associated with diagnosis of AN;
identifying, with the computing system, a second set of weighted values each corresponding to each of the received first set of patient responses from the first patient;
calculating, with the computing system, a second probability of diagnosis of bulimia nervosa (“BN”), based at least in part on modification of the received second set of patient responses by multiplication with the second set of weighted values and by subsequent addition of a second constant value associated with diagnosis of BN;
identifying, with the computing system, a third set of weighted values each corresponding to each of the received first set of patient responses from the first patient;
calculating, with the computing system, a third probability of diagnosis of binge-eating disorder (“BED”), based at least in part on modification of the received third set of patient responses by multiplication with the third set of weighted values and by subsequent addition of a third constant value associated with diagnosis of BED;
identifying, with the computing system, a fourth set of weighted values each corresponding to each of the received first set of patient responses from the first patient;
calculating, with the computing system, a fourth probability of diagnosis of obesity (“OB”), based at least in part on modification of the received fourth set of patient responses by multiplication with the fourth set of weighted values and by subsequent addition of a fourth constant value associated with diagnosis of OB;
identifying, with the computing system, a fifth set of weighted values each corresponding to each of the received first set of patient responses from the first patient;
calculating, with the computing system, a fifth probability of diagnosis of other specified feeding or eating disorder (“OSFED”), based at least in part on modification of the received fifth set of patient responses by multiplication with the fifth set of weighted values and by subsequent addition of a fifth constant value associated with diagnosis of OSFED;
identifying, with the computing system, which eating disorder the first patient is likely to have, based at least in part on the determined diagnosis and based at least in part on the calculated first through fifth probabilities;
identifying, with the computing system, suggested therapy techniques for the identified eating disorder; and
modifying, with the computing system, one or more of the first set of weighted values, the second set of weighted values, the third set of weighted values, the fourth set of weighted values, the fifth set of weighted values, the first constant value associated with diagnosis of AN, the second constant value associated with diagnosis of BN, the third constant value associated with diagnosis of BED, the fourth constant value associated with diagnosis of OB, or the fifth constant value associated with diagnosis of OSFED, based at least in part on one or more of the received first set of patient responses or a plurality of patient responses associated with a plurality of patients.

2. A method, comprising:
receiving, with a computing system and from a first patient, a first set of patient responses to a set of questions each having closed-ended answer options;
autonomously determining, with the computing system, a diagnosis of whether or not the first patient has an eating disorder, based at least in part on logistic regression analysis of the first set of patient responses; and
based on a determination that the first patient likely has an eating disorder, performing one or more of the following:
autonomously identifying, with the computing system, a first set of weighted values each corresponding to each of the received first set of patient responses from the first patient;
autonomously calculating, with the computing system, a first probability of diagnosis of a first eating disorder, based at least in part on modification of the received first set of patient responses by multiplication with the first set of weighted values and by subsequent addition of a first constant value associated with diagnosis of the first eating disorder;
autonomously identifying, with the computing system, a second set of weighted values each corresponding to each of the received first set of patient responses from the first patient;
autonomously calculating, with the computing system, a second probability of diagnosis of a second eating disorder, based at least in part on modification of the received second set of patient responses by multiplication with the second set of weighted values and by subsequent addition of a second constant value associated with diagnosis of the second eating disorder;
autonomously identifying, with the computing system, which eating disorder the first patient is likely to have, based at least in part on the determined diagnosis and based at least in part on the calculated first and second probabilities; and
displaying, with the computing system and on a display device, the identified eating disorder.

3. The method of claim 2, wherein each question is dichotomized such that a first set of answer options among its closed-ended answer options are assigned a first score while a second set of answer options among its closed-ended answer options are assigned a second score, wherein the first score is indicative of likelihood of patients selecting such answer options having an eating disorder, and wherein the second score is indicative of likelihood of patients selecting such answer options not having an eating disorder, wherein the method further comprises:
autonomously determining, with the computing system and for each dichotomized question among the set of dichotomized questions, whether a corresponding patient response among the first set of patient responses corresponds to the first set of answer options for that dichotomized question or corresponds to the second set of answer options for that dichotomized question;
wherein autonomously determining the diagnosis of whether or not the first patient has an eating disorder is further based at least in part on logistic regression analysis of one or more of the patient responses among the first set of patient responses corresponding to the first set of answer options that are assigned the first score or the patient responses among the first set of patient responses corresponding to the second set of answer options that are assigned the second score.

4. The method of claim 2, further comprising, based on the determination that the first patient likely has an eating disorder:
autonomously identifying, with the computing system, a third set of weighted values each corresponding to each of the received first set of patient responses from the first patient;
autonomously calculating, with the computing system, a third probability of diagnosis of a third eating disorder, based at least in part on modification of the received third set of patient responses by multiplication with the third set of weighted values and by subsequent addition of a third constant value associated with diagnosis of the third eating disorder;
autonomously identifying, with the computing system, a fourth set of weighted values each corresponding to each of the received first set of patient responses from the first patient;
autonomously calculating, with the computing system, a fourth probability of diagnosis of a fourth eating disorder, based at least in part on modification of the received fourth set of patient responses by multiplication with the fourth set of weighted values and by subsequent addition of a fourth constant value associated with diagnosis of the fourth eating disorder;
autonomously identifying, with the computing system, a fifth set of weighted values each corresponding to each of the received first set of patient responses from the first patient;
autonomously calculating, with the computing system, a fifth probability of diagnosis of a fifth eating disorder, based at least in part on modification of the received fifth set of patient responses by multiplication with the fifth set of weighted values and by subsequent addition of a fifth constant value associated with diagnosis of the fifth eating disorder; and
wherein autonomously identifying which eating disorder the first patient is likely to have comprises autonomously identifying, with the computing system, which eating disorder the first patient is likely to have, based at least in part on the determined diagnosis and based at least in part on the calculated first through fifth probabilities.

5. The method of claim 4, wherein the first eating disorder, the second eating disorder, the third eating disorder, the fourth eating disorder, and the fifth eating disorder each comprises one of anorexia nervosa (“AN”), bulimia nervosa (“BN”), binge-eating disorder (“BED”), obesity (“OB”), or other specified feeding or eating disorder (“OSFED”).

6. The method of claim 4, wherein the first patient is among a plurality of patients, wherein the method further comprises:
receiving, with the computing system, a plurality of sets of patient responses associated with the plurality of patients, an identified eating disorder associated with each patient among the plurality of patients, and a diagnosis of each patient among the plurality of patients performed by one or more clinicians, wherein the plurality of patient response comprises the first set of patient responses associated with the first patient;
autonomously comparing, with the computing system, the identified eating disorder associated with each patient among the plurality of patients with the received diagnosis of each patient performed by the one or more clinicians to determine whether the identified eating disorder matches the received diagnosis;
based on the comparison, autonomously analyzing, with the computing system, the plurality of sets of patient responses associated with the plurality of patients, the identified eating disorder associated with each patient among the plurality of patients, the diagnosis of each patient among the plurality of patients performed by the one or more clinicians, and the first through fifth set of weighted values to determine whether one or more of the first set of weighted values, the second set of weighted values, the third set of weighted values, the fourth set of weighted values, the fifth set of weighted values, the first constant value associated with diagnosis of the first eating disorder, the second constant value associated with diagnosis of the second eating disorder, the third constant value associated with diagnosis of the third eating disorder, the fourth constant value associated with diagnosis of the fourth eating disorder, or the fifth constant value associated with diagnosis of the fifth eating disorder are optimal or should be updated or modified; and
autonomously modifying, with the computing system, one or more of the first set of weighted values, the second set of weighted values, the third set of weighted values, the fourth set of weighted values, the fifth set of weighted values, the first constant value associated with diagnosis of the first eating disorder, the second constant value associated with diagnosis of the second eating disorder, the third constant value associated with diagnosis of the third eating disorder, the fourth constant value associated with diagnosis of the fourth eating disorder, or the fifth constant value associated with diagnosis of the fifth eating disorder, based at least in part on one or more of the comparison of the identified eating disorder associated with each patient among the plurality of patients with the received diagnosis of each patient performed by the one or more clinicians, the received plurality of sets of patient responses associated with the plurality of patients, or the analysis.

7. The method of claim 6, wherein at least one of autonomously identifying each of the first through fifth set of weighted values, autonomously calculating the first through fifth probability of diagnosis, autonomously identifying which eating disorder the first patient is likely to have, autonomously comparing the identified eating disorder associated with each patient among the plurality of patients with the received diagnosis of each patient performed by the one or more clinicians, or autonomously modifying the one or more of the first set of weighted values, the second set of weighted values, the third set of weighted values, the fourth set of weighted values, the fifth set of weighted values, the first constant value associated with diagnosis of the first eating disorder, the second constant value associated with diagnosis of the second eating disorder, the third constant value associated with diagnosis of the third eating disorder, the fourth constant value associated with diagnosis of the fourth eating disorder, or the fifth constant value associated with diagnosis of the fifth eating disorder is performed in near-real-time.

8. The method of claim 2, wherein the display device comprises one of a tablet computer, a smart phone, a mobile phone, a laptop computer, a desktop computer, or a monitor.

9. The method of claim 2, wherein displaying the identified eating disorder comprises displaying, with the computing system, the identified eating disorder in a software application (“app”) running on the display device.

10. The method of claim 2, wherein receiving the first set of patient responses comprises receiving, with the computing system and from a user device that receives input from the first patient, the first set of patient responses to the set of questions.

11. The method of claim 2, further comprising:
sending, with the computing system, a message to a user device associated with a medical practitioner, the message comprising the identified eating disorder associated with the first patient.

12. The method of claim 11, wherein the medical practitioner comprises one of a general medical practitioner, a primary care physician, a psychiatrist, a clinician, or a nurse.

13. The method of claim 11, wherein the user device associated with the medical practitioner comprises one of a tablet computer, a smart phone, a mobile phone, a laptop computer, or a desktop computer.

14. The method of claim 11, wherein the message further comprises suggested therapy techniques associated with the identified eating disorder associated with the first patient.

15. The method of claim 2, wherein the set of questions comprises a first category of questions, a second category of questions, and a third category of questions, wherein the first category of questions comprises questions regarding conditions including at least one of body-mass index (“BMI”), weight loss during the previous year, or self-induced vomiting, wherein the second category of questions comprises questions regarding behavior including at least one of eating patterns, dieting, weighing one's self, isolation from friends and family, or avoiding activities, wherein the third category of questions comprises questions regarding thoughts including at least one of being afraid of losing control over eating, thoughts about food, believing one's self to be fat when others call one too thin, or reaction to weight gain, wherein the first set of weighted values are differently defined based on differences among the first category of questions, the second category of questions, and the third category of questions.

16. A system, comprising:
a computing system, comprising:
at least one first processor; and
a first non-transitory computer readable medium communicatively coupled to the at least one first processor, the first non-transitory computer readable medium having stored thereon computer software comprising a first set of instructions that, when executed by the at least one first processor, causes the computing system to:
receive, from a first patient, a first set of patient responses to a set of questions each having closed-ended answer options;
autonomously determine a diagnosis of whether or not the first patient has an eating disorder, based at least in part on logistic regression analysis of the first set of patient responses; and
based on a determination that the first patient likely has an eating disorder, perform one or more of the following:
autonomously identify a first set of weighted values each corresponding to each of the received first set of patient responses from the first patient;
autonomously calculate a first probability of diagnosis of a first eating disorder, based at least in part on modification of the received first set of patient responses by multiplication with the first set of weighted values and by subsequent addition of a first constant value associated with diagnosis of the first eating disorder;
autonomously identify a second set of weighted values each corresponding to each of the received first set of patient responses from the first patient;
autonomously calculate a second probability of diagnosis of a second eating disorder, based at least in part on modification of the received second set of patient responses by multiplication with the second set of weighted values and by subsequent addition of a second constant value associated with diagnosis of the second eating disorder;
autonomously identify which eating disorder the first patient is likely to have, based at least in part on the determined diagnosis and based at least in part on the calculated first and second probabilities; and
display, on a display device, the identified eating disorder.

17. The system of claim 16, wherein each question is dichotomized such that a first set of answer options among its closed-ended answer options are assigned a first score while a second set of answer options among its closed-ended answer options are assigned a second score, wherein the first score is indicative of likelihood of patients selecting such answer options having an eating disorder, and wherein the second score is indicative of likelihood of patients selecting such answer options not having an eating disorder, wherein the first set of instructions, when executed by the at least one first processor, further causes the computing system to:
autonomously determine, for each dichotomized question among the set of dichotomized questions, whether a corresponding patient response among the first set of patient responses corresponds to the first set of answer options for that dichotomized question or corresponds to the second set of answer options for that dichotomized question;
wherein autonomously determining the diagnosis of whether or not the first patient has an eating disorder is further based at least in part on logistic regression analysis of one or more of the patient responses among the first set of patient responses corresponding to the first set of answer options that are assigned the first score or the patient responses among the first set of patient responses corresponding to the second set of answer options that are assigned the second score.

18. The system of claim 16, wherein, based on the determination that the first patient likely has an eating disorder, the first set of instructions, when executed by the at least one first processor, further causes the computing system to:
autonomously identify a third set of weighted values each corresponding to each of the received first set of patient responses from the first patient;
autonomously calculate a third probability of diagnosis of a third eating disorder, based at least in part on modification of the received third set of patient responses by multiplication with the third set of weighted values and by subsequent addition of a third constant value associated with diagnosis of the third eating disorder;
autonomously identify a fourth set of weighted values each corresponding to each of the received first set of patient responses from the first patient;
autonomously calculate a fourth probability of diagnosis of a fourth eating disorder, based at least in part on modification of the received fourth set of patient responses by multiplication with the fourth set of weighted values and by subsequent addition of a fourth constant value associated with diagnosis of the fourth eating disorder;
autonomously identify a fifth set of weighted values each corresponding to each of the received first set of patient responses from the first patient;
autonomously calculate a fifth probability of diagnosis of a fifth eating disorder, based at least in part on modification of the received fifth set of patient responses by multiplication with the fifth set of weighted values and by subsequent addition of a fifth constant value associated with diagnosis of the fifth eating disorder; and
wherein autonomously identifying which eating disorder the first patient is likely to have comprises autonomously identifying which eating disorder the first patient is likely to have, based at least in part on the determined diagnosis and based at least in part on the calculated first through fifth probabilities.

19. The system of claim 18, wherein the first eating disorder, the second eating disorder, the third eating disorder, the fourth eating disorder, and the fifth eating disorder each comprises one of anorexia nervosa (“AN”), bulimia nervosa (“BN”), binge-eating disorder (“BED”), obesity (“OB”), or other specified feeding or eating disorder (“OSFED”).

20. The system of claim 18, wherein the first patient is among a plurality of patients, wherein the first set of instructions, when executed by the at least one first processor, further causes the computing system to:
receive a plurality of sets of patient responses associated with the plurality of patients, an identified eating disorder associated with each patient among the plurality of patients, and a diagnosis of each patient among the plurality of patients performed by one or more clinicians, wherein the plurality of patient response comprises the first set of patient responses associated with the first patient;
autonomously compare the identified eating disorder associated with each patient among the plurality of patients with the received diagnosis of each patient performed by the one or more clinicians to determine whether the identified eating disorder matches the received diagnosis;
based on the comparison, autonomously analyze the plurality of sets of patient responses associated with the plurality of patients, an identified eating disorder associated with each patient among the plurality of patients, a diagnosis of each patient among the plurality of patients performed by the one or more clinicians, and the first through fifth set of weighted values to determine whether one or more of the first set of weighted values, the second set of weighted values, the third set of weighted values, the fourth set of weighted values, the fifth set of weighted values, the first constant value associated with diagnosis of the first eating disorder, the second constant value associated with diagnosis of the second eating disorder, the third constant value associated with diagnosis of the third eating disorder, the fourth constant value associated with diagnosis of the fourth eating disorder, or the fifth constant value associated with diagnosis of the fifth eating disorder are optimal or should be updated or modified; and
autonomously modify one or more of the first set of weighted values, the second set of weighted values, the third set of weighted values, the fourth set of weighted values, the fifth set of weighted values, the first constant value associated with diagnosis of the first eating disorder, the second constant value associated with diagnosis of the second eating disorder, the third constant value associated with diagnosis of the third eating disorder, the fourth constant value associated with diagnosis of the fourth eating disorder, or the fifth constant value associated with diagnosis of the fifth eating disorder, based at least in part on one or more of the comparison of the identified eating disorder associated with each patient among the plurality of patients with the received diagnosis of each patient performed by the one or more clinicians, the received plurality of sets of patient responses associated with the plurality of patients, or the analysis.

21. A method, comprising:
receiving, with a computing system, a plurality of sets of patient responses to a set of questions each having closed-ended answer options, the plurality of sets of patient responses being associated with a plurality of patients, an identified eating disorder associated with each patient among the plurality of patients, and a diagnosis of each patient among the plurality of patients performed by one or more clinicians;
autonomously comparing, with the computing system, the identified eating disorder associated with each patient among the plurality of patients with the received diagnosis of each patient performed by the one or more clinicians to determine whether the identified eating disorder matches the received diagnosis; and
autonomously modifying, with the computing system, one or more of a first set of weighted values, a second set of weighted values, a third set of weighted values, a fourth set of weighted values, a fifth set of weighted values, a first constant value associated with diagnosis of a first eating disorder, a second constant value associated with diagnosis of a second eating disorder, a third constant value associated with diagnosis of a third eating disorder, a fourth constant value associated with diagnosis of a fourth eating disorder, or a fifth constant value associated with diagnosis of a fifth eating disorder, based at least in part on one or more of the comparison of the identified eating disorder associated with each patient among the plurality of patients with the received diagnosis of each patient performed by the one or more clinicians or the received plurality of patient responses associated with the plurality of patients.

22. The method of claim 21, further comprising:
based on the comparison, autonomously analyzing, with the computing system, the plurality of sets of patient responses associated with the plurality of patients, the identified eating disorder associated with each patient among the plurality of patients, the diagnosis of each patient among the plurality of patients performed by the one or more clinicians, and the first through fifth set of weighted values to determine whether one or more of the first set of weighted values, the second set of weighted values, the third set of weighted values, the fourth set of weighted values, the fifth set of weighted values, the first constant value associated with diagnosis of the first eating disorder, the second constant value associated with diagnosis of the second eating disorder, the third constant value associated with diagnosis of the third eating disorder, the fourth constant value associated with diagnosis of the fourth eating disorder, or the fifth constant value associated with diagnosis of the fifth eating disorder are optimal or should be updated or modified.

23. The method of claim 21, wherein the first set of weighted values, the second set of weighted values, the third set of weighted values, the fourth set of weighted values, and the fifth set of weighted values each corresponds to each of the received plurality of sets of patient responses.

24. The method of claim 21, wherein the first eating disorder, the second eating disorder, the third eating disorder, the fourth eating disorder, and the fifth eating disorder each comprises one of anorexia nervosa (“AN”), bulimia nervosa (“BN”), binge-eating disorder (“BED”), obesity (“OB”), or other specified feeding or eating disorder (“OSFED”).

25. The method of claim 21, wherein at least one of autonomously comparing the identified eating disorder associated with each patient among the plurality of patients with the received diagnosis of each patient performed by the one or more clinicians or autonomously modifying the one or more of the first set of weighted values, the second set of weighted values, the third set of weighted values, the fourth set of weighted values, the fifth set of weighted values, the first constant value associated with diagnosis of the first eating disorder, the second constant value associated with diagnosis of the second eating disorder, the third constant value associated with diagnosis of the third eating disorder, the fourth constant value associated with diagnosis of the fourth eating disorder, or the fifth constant value associated with diagnosis of the fifth eating disorder is performed in near-real-time.

26. The method of claim 21, wherein the set of questions comprises a first category of questions, a second category of questions, and a third category of questions, wherein the first category of questions comprises questions regarding conditions including at least one of body-mass index (“BMI”), weight loss during the previous year, or self-induced vomiting, wherein the second category of questions comprises questions regarding behavior including at least one of eating patterns, dieting, weighing one's self, isolation from friends and family, or avoiding activities, wherein the third category of questions comprises questions regarding thoughts including at least one of being afraid of losing control over eating, thoughts about food, believing one's self to be fat when others call one too thin, or reaction to weight gain, wherein the first set of weighted values are differently defined based on differences among the first category of questions, the second category of questions, and the third category of questions.

27. A system, comprising:
a computing system, comprising:
at least one first processor; and
a first non-transitory computer readable medium communicatively coupled to the at least one first processor, the first non-transitory computer readable medium having stored thereon computer software comprising a first set of instructions that, when executed by the at least one first processor, causes the computing system to:
receive a plurality of sets of patient responses to a set of questions each having closed-ended answer options, the plurality of sets of patient responses being associated with a plurality of patients, an identified eating disorder associated with each patient among the plurality of patients, and a diagnosis of each patient among the plurality of patients performed by one or more clinicians;
autonomously compare the identified eating disorder associated with each patient among the plurality of patients with the received diagnosis of each patient performed by the one or more clinicians to determine whether the identified eating disorder matches the received diagnosis; and
autonomously modify one or more of a first set of weighted values, a second set of weighted values, a third set of weighted values, a fourth set of weighted values, a fifth set of weighted values, a first constant value associated with diagnosis of a first eating disorder, a second constant value associated with diagnosis of a second eating disorder, a third constant value associated with diagnosis of a third eating disorder, a fourth constant value associated with diagnosis of a fourth eating disorder, or a fifth constant value associated with diagnosis of a fifth eating disorder, based at least in part on one or more of the comparison of the identified eating disorder associated with each patient among the plurality of patients with the received diagnosis of each patient performed by the one or more clinicians or the received plurality of patient responses associated with the plurality of patients.

28. The system of claim 27, wherein the first set of weighted values, the second set of weighted values, the third set of weighted values, the fourth set of weighted values, and the fifth set of weighted values each corresponds to each of the received plurality of sets of patient responses.

29. The system of claim 27, wherein the first eating disorder, the second eating disorder, the third eating disorder, the fourth eating disorder, and the fifth eating disorder each comprises one of anorexia nervosa (“AN”), bulimia nervosa (“BN”), binge-eating disorder (“BED”), obesity (“OB”), or other specified feeding or eating disorder (“OSFED”).

30. The system of claim 27, wherein at least one of autonomously comparing the identified eating disorder associated with each patient among the plurality of patients with the received diagnosis of each patient performed by the one or more clinicians or autonomously modifying the one or more of the first set of weighted values, the second set of weighted values, the third set of weighted values, the fourth set of weighted values, the fifth set of weighted values, the first constant value associated with diagnosis of the first eating disorder, the second constant value associated with diagnosis of the second eating disorder, the third constant value associated with diagnosis of the third eating disorder, the fourth constant value associated with diagnosis of the fourth eating disorder, or the fifth constant value associated with diagnosis of the fifth eating disorder is performed in near-real-time.

31. The system of claim 27, wherein the set of questions comprises a first category of questions, a second category of questions, and a third category of questions, wherein the first category of questions comprises questions regarding conditions including at least one of body-mass index (“BMI”), weight loss during the previous year, or self-induced vomiting, wherein the second category of questions comprises questions regarding behavior including at least one of eating patterns, dieting, weighing one's self, isolation from friends and family, or avoiding activities, wherein the third category of questions comprises questions regarding thoughts including at least one of being afraid of losing control over eating, thoughts about food, believing one's self to be fat when others call one too thin, or reaction to weight gain, wherein the first set of weighted values are differently defined based on differences among the first category of questions, the second category of questions, and the third category of questions.
